(19) United States
(12) Patent Application Publication
(10) Pub. No.: US 2022/0411880 A1
(43) Pub. Date: Dec. 29, 2022

(54) METHODS AND COMPOSITIONS FOR DIAGNOSING AND TREATING CHRONIC MYELOMONOCYTIC LEUKEMIA (CMML)

(71) Applicants: INSERM (INSTITUT NATIONAL DE LA SANTÉET DE LA RECHERCHE MÉDICALE), Paris (FR); UNIVERSITE COTE D'AZUR, Nice (FR); MEMORIAL SLOAN KETTERING CANCER CENTER, New York, NY (US); THE TRUSTEES OF COLUMBIA UNIVERSITY IN THE CITY OF NEW YORK, New York, NY (US)

(72) Inventors: Laurent YVAN-CHARVET, Nice (FR); Manon VIAUD, Nice (FR); Alan R. TALL, New York, NY (US); Ross L. LEVINE, New York, NY (US); Omar ABDEL-WAHAB, New York, NY (US)

(21) Appl. No.: 17/778,729

(22) PCT Filed: Nov. 20, 2020
(86) PCT No.: PCT/EP2020/082889

§ 371 Date: May 20, 2022

(30) Foreign Application Priority Data
Nov. 21, 2019 (EP) 19306501.8

(51) Int. Cl. C12Q 1/6886; A61K 31/724; C07K 16/28; A61K 38/17

(52) U.S. Cl. CPC C12Q 1/6886; A61K 31/724; C07K 16/2866; A61K 38/1709; C12Q 2600/156; C12Q 2600/158; C12Q 2600/118

(57) ABSTRACT

In the present invention, inventors have used high throughput sequencing to identify novel mutations in ABCA1 in CM ML patient samples. Further studies in a mouse model of myelomonocytic leukemia driven by hematopoietic Tet2 deficiency have shown that these somatic mutations abrogate the tumor suppressor function of WT ABCA1, resulting in the failure to suppress canonical IL3-receptor beta signaling-driven myelopoiesis. The loss of the myelo-suppressive function of ABCA1 mutants can be overcome by raising HDL levels through overexpression of the human apolipoprotein A-1 (apoA-1) transgene. Inventors have also shown that both IL-3Rbeta blocking antibody and cyclodextrin prevented the proliferation of ABCA1 mutant-transduced Tet2 deficient BM cells similar to the effect of ABCA1-WT overexpression. Accordingly, the invention relates to a method for predicting the survival time of a subject NI suffering from CM ML comprising the step identifying at least one ABCA1 and to a method for treating said subject with HDL/ABCA recombinant (ApoA-1); cylodextrin and/or anti-IL-3Rbeta antibody.

### FIELD OF THE INVENTION

The invention is in the field of oncology. More particularly, the invention relates to methods and compositions to treat Chronic MyeloMonocytic Leukemia (CMML).

### BACKGROUND OF THE INVENTION

Most human adult cancers develop through a multistep acquisition of a wide range of somatic mutations that initiate or maintain self-renewal of the malignant clone. The last decade has seen the elucidation of the somatic mutational landscape of many solid tumors and hematologic malignancies (1, 2). These mutations are referenced in the Catalog of Somatic Mutations In Cancer (COSMIC) and The Cancer Genome Atlas (TCGA), and may provide potential novel insights into mechanisms underlying cancer. In myeloid malignancies, hematopoietic stem and progenitor cells (HSPCs) acquire specific combinations of leukemia disease alleles required to promote hematopoietic transformation (3, 4). Recent studies have shown that mutations in a small number of genes, including loss-of-function mutations in TET2 are common in the elderly and provide a proliferative advantage to hematopoietic stem cells giving rise to clonal hematopoiesis (5). Clonal hematopoiesis mutations are associated with about a 10-fold increase in risk of developing a hematological malignancy including myeloproliferative disorders and leukemias and a 2-3-fold risk of developing atherosclerotic cardiovascular disease.

Increased high-density lipoprotein (HDL) levels are well known to be associated with a reduced risk of cardio vascular diseases (CVD). Interestingly, a recent meta-analysis of randomized controlled trials of lipid-altering therapies revealed that for every 10 mg/dL increase in plasma HDL-cholesterol level among trial participants, there was a 36% lower risk of cancer incidence during >625,000 person-years of follow-up and >8,000 incident cancers (6). While not establishing causation, this association suggests that HDL may be linked to tumor cell biology in humans. The ability of HDL and its apolipoproteins to promote efflux of cholesterol from cells depends in part on the ATP-binding cassette transporters ABCA1 and ABCG1 but can also be mediated by scavenger receptor B1 and passive efflux pathways (7).

Mice with defective cholesterol efflux in hematopoietic cells develop progressive myeloid expansion with an underlying dramatic HSPC expansion in the BM, an enhanced IL-3/GM-CSF signaling pathway and marked extramedullary hematopoiesis (13-16). We also demonstrated that HDL raising therapies could limit Mp1-W515L and Flt3-ITD-driven myeloproliferative disorders (17).

Chronic Myelomonocytic Leukemia (CMML) is typically a disease of the elderly with few treatment options. Recent studies in CMML patients have shown changes reminiscent of those observed in mice with defective cholesterol efflux in hematopoietic cells including: 1) frequently mutated tumor suppressor genes encoding regulators of GM-CSF signaling (RAS, CBL), 2) hypersensitivity of myeloid progenitor to GM-CSF and 3) a proportion of ‘classical’ CD14+CD16− monocytes >94% (18). In addition, these patients often have mutations in genes associated with clonal hematopoiesis including TET2 and ASXL1.

Accordingly, there is a need to understand the role of defective cholesterol efflux in hematopoietic cell, identify new biomarkers and new therapeutically tools to treat CMML.

### SUMMARY OF THE INVENTION

The invention relates to a method for predicting the survival time of a subject suffering from chronic myelomonocytic leukemia (CMML) comprising the steps of i) identifying at least one mutation in ATP-binding cassette A1 (ABCA1) at gene, ARN or protein level in a biological sample obtained from the subject; and ii) concluding that the subject will have a short survival time when at least one mutation in ABCA1 at gene, ARN or protein level is identified or concluding that the subject will have a long survival time when any mutation is not identified in ABCA1 at gene, ARN or protein level. In particular, the present invention is defined by the claims.

### DETAILED DESCRIPTION OF THE INVENTION

Inventors have identified five somatic missense mutations in ABCA1 in 26 patients with CMML. These mutations conferred a proliferative advantage to monocytic leukemia cell lines in vitro. In vivo inactivation of ABCA1 or expression of ABCA1 mutants in hematopoietic cells in the setting of Tet2 loss (which commonly occurs in hematological malignancies including CMML) demonstrated a myelo-suppressive function of ABCA1 that limited the development of a fully penetrant myeloproliferative disorder. Mechanistically, ABCA1 mutations impaired the tumor suppressor functions of WT ABCA1 in myelomonocytic leukemia by increasing the IL3-receptor beta canonical pathway signaling via MAPK and JAK2 and subsequent metabolic reprogramming. Overexpression of a human apolipoprotein A-1 transgene to promote cholesterol efflux dampened myeloproliferation. These findings identify novel somatic mutations in ABCA1 that subvert its anti-proliferative and cholesterol efflux functions and permit the progression of CMML. Therapeutic increases in HDL bypassed these defects and restored normal hematopoiesis.

Accordingly, in a first aspect, the invention relates to a method for diagnosing a chronic myelomonocytic leukemia (CMML) in a subject, wherein said method comprising a step of detecting a mutation in a in ATP-binding cassette A1 (ABCA1) gene, ARN or protein level in a biological sample obtained from said subject, wherein the presence of a mutation is indicative of a CMML.

In a second aspect, the invention relates to a method for predicting the survival time of a subject suffering from chronic myelomonocytic leukemia (CMML) comprising the steps of i) identifying at least one mutation in ATP-binding cassette A1 (ABCA1) at gene, ARN or protein level in a biological sample obtained from the subject; and ii) concluding that the subject will have a short survival time when at least one mutation in ABCA1 is identified or concluding that the subject will have a long survival time when any mutation is not identified in ABCA1.

As used herein, the term “predicting” means that the subject to be analyzed by the method of the invention is allocated either into the group of subjects who will have or develop chronic myelomonocytic leukemia (CMML) or into a group of subjects who will not have or develop CMML. Having or developing CMML referred to in accordance with the invention, particularly, means that the subject will have higher risk to have or develop CMML. Typically, said risk is elevated as compared to the average risk in a cohort of subjects suffering from CMML.

In the context of the invention, the risk of having the CMML in a subject susceptible to suffer from CMML be predicted. The term “predicting the risk”, as used herein, refers to assessing the probability according to which the patient as referred to herein will have or develop CMML.

As will be understood by those skilled in the art, such an assessment is usually not intended to be correct for 100% of the subjects to be investigated. The term, however, requires that prediction can be made for a statistically significant portion of subjects in a proper and correct manner. Whether a portion is statistically significant can be determined without further ado by the person skilled in the art using various well known statistic evaluation tools, e.g., determination of confidence intervals, p-value determination, Student's t-test, Mann-Whitney test etc. Details are found in Dowdy and Wearden, Statistics for Research, John Wiley & Sons, New York 1983. Preferred confidence intervals are at least 90%, at least 95%, at least 97%, at least 98% or at least 99%. The p-values are, preferably, 0.1, 0.05, 0.01, 0.005, or 0.0001. Preferably, the probability envisaged by the invention allows that the prediction of an increased risk will be correct for at least 60%, at least 70%, at least 80%), or at least 90% of the subjects of a given cohort or population.

As used herein, the term “subject” denotes a mammal, such as a rodent, a feline, a canine, and a primate. Particularly, the subject according to the invention is a human. More particularly, the subject according to the invention has or is susceptible to have chronic myelomonocytic leukemia (CMML).

As used herein, the term “Chronic myelomonocytic leukaemia (CMML)” is a type of leukemia, which are cancers of the blood-forming cells of the bone marrow. It is a rare disorder with an estimated incidence of 1 case per 100 000 persons per year. Median age at presentation is 70 years, and presenting manifestations may include those of bone marrow failure and systemic symptoms. Hepatomegaly and splenomegaly are found in some patients, and the white blood cell count is typically increased. CMML was reclassified by the World Health Organization (WHO) as a myelodysplastic/myeloproliferative neoplasm (MDS/MPN) (Jaffe et al., 2001)

As used herein, the term “ABCA1” also known as the cholesterol efflux regulatory protein (CERP) is a protein which in humans is encoded by the ABCA1 gene. ABCA1 refers to ATP-binding cassette A1 and is a major regulator of cellular cholesterol and phospholipid homeostasis.

The naturally occurring murin ABCA1 gene has a nucleotide sequence as shown in Genbank Accession numbers NM_013454. The naturally occurring human ABCA1 protein has an aminoacid sequence as shown in Genbank Accession numbers NP_038482.

The naturally occurring human ABCA1 gene has a nucleotide sequence as shown in Genbank Accession numbers NM_005502. The naturally occurring human ABCA1 protein has an aminoacid sequence as shown in Genbank Accession numbers NP_005493.

As used herein, the term “gene” has its general meaning in the art and refers to means a DNA sequence that codes for or corresponds to a particular sequence of amino acids which comprise all or part of one or more proteins or enzymes, and may or may not include regulatory DNA sequences, such as promoter sequences, which determine for example the conditions under which the gene is expressed.

As used herein the “allele” has its general meaning in the art and refers to an alternative form of a gene (one member of a pair) that is located at a specific position on a specific chromosome which, when translated result in functional or dysfunctional (including nonexistent) gene products.

As used herein, the term “protein” has its general meaning in the art and refers to one or more long chains of amino acid residues which comprise all or part of one or more proteins or enzymes. Typically, ABCA1 protein mediates the efflux of cholesterol and phospholipids to lipid-poor apolipoproteins (apo-A1 and apoE), which then form nascent high-density lipoproteins (HDL).

As used herein, the term “biological sample” refers to any sample obtained from a subject, such as a serum sample, a plasma sample, a urine sample, a blood sample, a lymph sample, bone marrow sample, or a tissue biopsy. In a particular embodiment, biological sample for the determination of an expression level include samples such as a blood sample or a urine sample, lymph sample, or a biopsy.

In a particular embodiment, the biological sample is a tissue biopsy.

In a particular embodiment, the biological sample is a bone marrow sample.

In a particular embodiment, the biological sample is a blood sample, more particularly, peripheral blood mononuclear cells (PBMC). Typically, these cells can be extracted from whole blood using Ficoll, a hydrophilic polysaccharide that separates layers of blood, with the PBMC forming a cell ring under a layer of plasma. Additionally, PBMC can be extracted from whole blood using a hypotonic lysis, which will preferentially lyse red blood cells. Such procedures are known to the experts in the art.

Inventors have shown that five somatic missense mutations in ABCA1 in 26 patients with CM ML leading to ABCA1-P711L, ABCA1-A1291T, ABCA1-G1421R, ABCA1-P1423S and ABCA1-A2011T. These mutations displayed reductions in anti-proliferative activity, compared to ABCA1-WT.

In a particular embodiment, the method according to the invention, wherein the mutations are located within the coding region of the ABCA1 gene.

In a particular embodiment, the method according to the invention, wherein the mutation is ABCA1-P711L in the ABCA1 protein.

In a particular embodiment, the method according to the invention, wherein the mutation is ABCA1-A1291T in the ABCA1 protein.

In a particular embodiment, the method according to the invention, wherein the mutation is ABCA1-G1421R in the ABCA1 protein.

In a particular embodiment, the method according to the invention, wherein the mutation is ABCA1-P1423S in the ABCA1 protein.

In a particular embodiment, the method according to the invention, wherein the mutation is ABCA1-A2011T in the ABCA1 protein.

In a particularly embodiment, the 5 mutations as described above are identified simultaneously, separately or sequentially in a biological sample.

In a particular embodiment, the invention relates to a method for diagnosing a chronic myelomonocytic leukemia (CMML) in a subject, said method comprising a step of detecting a ABCA1-P711L, ABCA1-A1291T, ABCA1-G1421R, ABCA1-P1423S and/or ABCA1-A2011T mutation in a in ATP-binding cassette A1 (ABCA1) gene, ARN or protein level in a biological sample obtained from said subject, wherein the presence of a mutation is indicative of a CMML.

In further embodiment, the invention relates to a method for predicting the survival time of a subject suffering from chronic myelomonocytic leukemia (CMML) comprising the steps of i) identifying ABCA1-P711L, ABCA1-A1291T, ABCA1-G1421R, ABCA1-P1423S and/or ABCA1-A2011T mutation in ATP-binding cassette A1 (ABCA1) at protein level in a biological sample obtained from the subject; and ii) concluding that the subject will have a short survival time when at least one mutation in ABCA1 is identified or concluding that the subject will have a long survival time when any mutation is identified in ABCA1.

Accordingly, the present invention also relates to a method for predicting the risk of having or developing CMML in a subject in need thereof, comprising the step of detecting ABCA1 single nucleotide polymorphism (SNP) in a biological sample obtained from said subject.

In a further aspect, the present invention relates to a method for predicting the risk of having or developing CMML in a subject in need thereof, comprising the step of determining the expression level of mutants ABCA1 and/or detecting ABCA1 SNP in a biological sample obtained from said subject.

In a particular embodiment, the invention relates to a method for predicting the risk of having or developing CMML in a subject in need thereof, comprising the steps of: i) determining the expression level of mutants ABCA1 protein and/or detecting ABCA1 SNP in a biological sample obtained from said subject, ii) comparing the expression level determined at step i) with a predetermined reference value and iii) concluding that the subject is at risk of having or developing CMML when the expression level determined at step i) is lower than the predetermined reference value and/or when the ABCA1 SNP is detected, or concluding that the patient is not at risk of having or developing CMML when the expression level determined at step i) is higher than the predetermined reference value and/or when the ABCA1 SNP is not detected.

In a particular embodiment, the method according to the invention, further comprising the steps of: i) identifying at least one mutation in the ABCA1 gene and/or protein; ii) concluding that the subject is at risk of having or developing CMML when at least one mutation is identified.

In a particular embodiment, the method according to the invention, further comprising the steps of: i) identifying at least one mutation in the ABCA1 gene and/or protein; ii) concluding that the subject is susceptible to have or having a short survival time when at least one mutation is identified.

As used herein, the term “mutation” has its general meaning in the art and refers to any detectable change in genetic material, e.g. DNA, RNA, cDNA, or any process, mechanism, or result of such a change. This includes gene mutations, in which the structure (e.g. DNA sequence) of a gene is altered, any gene or DNA arising from any mutation process, and any expression product (e.g. protein or enzyme) expressed by a modified gene or DNA sequence. Mutations include deletion, insertion or substitution of one or more nucleotides. The mutation may occur in the coding region of a gene (i.e. in exons), in introns, or in the regulatory regions (e.g. enhancers, response elements, suppressors, signal sequences, polyadenylation sequences, promoters) of the gene. Generally a mutation is identified in a subject by comparing the sequence of a nucleic acid or polypeptide expressed by said subject with the corresponding nucleic acid or polypeptide expressed in a control population. Where the mutation is within the gene coding sequence, the mutation may be a “missense” mutation, where it replaces one amino acid with another in the gene product, or a “non sense” mutation, where it replaces an amino acid codon with a stop codon. A mutation may also occur in a splicing site where it creates or destroys signals for exon-intron splicing and thereby lead to a gene product of altered structure. A mutation in the genetic material may also be “silent”, i.e. the mutation does not result in an alteration of the amino acid sequence of the expression product.

As used herein, the term “homozygous” refers to an individual possessing two copies of the same allele. As used herein, the term “homozygous mutant” refers to an individual possessing two copies of the same allele, such allele being characterized as the mutant form of a gene.

As used herein, the term “heterozygous” refers to an individual possessing two different alleles of the same gene, i.e. an individual possessing two different copies of an allele, such alleles are characterized as mutant forms of a gene. In a particular embodiment, the mutation allows to a truncated protein. Typically, truncated protein refers to a protein shortened by a mutation which specifically induces premature termination of messenger RNA translation.

As used herein, the term “single nucleotide polymorphism (SNP)” refers to is a single basepair variation in a nucleic acid sequence of ABCA1 gene. Polymorphisms can be referred to, for instance, by the nucleotide position at which the variation exists, by the change in amino acid sequence caused by the nucleotide variation, or by a change in some other characteristic of the nucleic acid molecule that is linked to the variation {e.g., an alteration of a secondary structure such as a stem-loop, or an alteration of the binding affinity of the nucleic acid for associated molecules, such as polymerases, RNases, and so forth). For example, the SNP in the context of the invention is missense mutation leading to the ABCA1-P711L, ABCA1-A1291T, ABCA1-G1421R, ABCA1-P1423S and ABCA1-A2011T in ABCA1.

In the methods according to the present the invention, the presence or absence of a SNP can be determined by nucleic acid sequencing, PCR analysis or any genotyping method known in the art such as the method described in the example. Examples of such methods include, but are not limited to, chemical assays such as allele specific hybridization (DASH), pyrosequencing, molecular beacons, SNP microarrays, restriction fragment length polymorphism (RFLP), flap endonuclease (FEN), single strand conformation polymorphism, temperature gradient gel electrophoresis (TGGE), denaturing high performance liquid chromatography (DHPLC), high-resolution melting of the entire amplicon, and DNA mismatch-binding proteins. primer extension, allele specific oligonucleotide ligation, sequencing, enzymatic cleavage, flap endonuclease discrimination; and detection methods such as fluorescence, chemiluminescence, and mass spectrometry.

For example, the presence or absence of said polymorphism may be detected in a DNA sample, preferably after amplification. For instance, the isolated DNA may be subjected to couple reverse transcription and amplification, such as reverse transcription and amplification by polymerase chain reaction (RT-PCR), using specific oligonucleotide primers that are specific for the polymorphism or that enable amplification of a region containing the polymorphism. According to a first alternative, conditions for primer annealing may be chosen to ensure specific reverse transcription (where appropriate) and amplification; so that the appearance of an amplification product be a diagnostic of the presence of the polymorphism according to the invention. Otherwise, DNA may be amplified, after which a mutated site may be detected in the amplified sequence by hybridization with a suitable probe or by direct sequencing, or any other appropriate method known in the art.

Currently numerous strategies for genotype analysis are available (Antonarakis et al., 1989; Cooper et al., 1991; Grompe, 1993). Briefly, the nucleic acid molecule may be tested for the presence or absence of a restriction site. When a base polymorphism creates or abolishes the recognition site of a restriction enzyme, this allows a simple direct PCR genotype the polymorphism. Further strategies include, but are not limited to, direct sequencing, restriction fragment length polymorphism (RFLP) analysis; hybridization with allele-specific oligonucleotides (ASO) that are short synthetic probes which hybridize only to a perfectly matched sequence under suitably stringent hybridization conditions; allele specific PCR; PCR using mutagenic primers; ligase-PCR, HOT cleavage; denaturing gradient gel electrophoresis (DGGE), temperature denaturing gradient gel electrophoresis (TGGE), single-stranded conformational polymorphism (SSCP) and denaturing high performance liquid chromatography (Kuklin et al., 1997). Direct sequencing may be accomplished by any method, including without limitation chemical sequencing, using the Maxam-Gilbert method; by enzymatic sequencing, using the Sanger method; mass spectrometry sequencing; pyrosequencing; sequencing using a chip-based technology and real-time quantitative PCR. Preferably, DNA from a patient is first subjected to amplification by polymerase chain reaction (PCR) using specific amplification primers. However several other methods are available, allowing DNA to be studied independently of PCR, such as the rolling circle amplification (RCA), the InvaderTMassay, or oligonucleotide ligation assay (OLA). OLA may be used for revealing base polymorphisms. According to this method, two oligonucleotides are constructed that hybridize to adjacent sequences in the target nucleic acid, with the join sited at the position of the polymorphism. DNA ligase will covalently join the two oligonucleotides only if they are perfectly hybridized to one of the allele. Oligonucleotide probes or primers may contain at least 10, 15, 20 or 30 nucleotides. Their length may be shorter than 400, 300, 200 or 100 nucleotides.

According to the invention, the determination of the presence or absence of said SNP may also be determined by detection or not of the mutated protein by any method known in the art. The presence of the protein of interest may be detected using standard electrophoretic and immunodiagnostic techniques, including immunoassays such as competition, direct reaction, or sandwich type assays. Such assays include, but are not limited to, Western blots; agglutination tests; enzyme-labelled and mediated immunoassays, such as ELISAs; biotin/avidin type assays; radioimmunoassays; immunoelectrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith. Labels are known in the art that generally provide (either directly or indirectly) a signal. As used herein, the term “labelled” with regard to the antibody or aptamer, is intended to encompass direct labelling of the antibody or aptamer by coupling (i.e., physically linking) a detectable substance, such as a radioactive agent or a fluorophore (e.g. fluorescein isothiocyanate (FITC) or phycoerythrin (PE) or indocyanine (Cy5), to the antibody or aptamer, as well as indirect labelling of the probe or antibody (e.g., horseradish peroxidise, HRP) by reactivity with a detectable substance. An antibody or aptamer may be also labelled with a radioactive molecule by any method known in the art. For example, radioactive molecules include but are not limited radioactive atom for scintigraphic studies such as 1123, 1124, In111, Re186 and Re188. The aforementioned assays generally involve separation of unbound protein in a liquid phase from a solid phase support to which antigen-antibody complexes are bound. Solid supports which may be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, etc.

More particularly, an ELISA method may be used, wherein the wells of a microtiter plate are coated with an antibody against the protein to be tested. A biological sample containing or suspected of containing the marker protein is then added to the coated wells. After a period of incubation sufficient to allow the formation of antibody-antigen complexes, the plate (s) can be washed to remove unbound moieties and a detectably labelled secondary binding molecule added. The secondary binding molecule is allowed to react with any captured sample marker protein, the plate washed and the presence of the secondary binding molecule detected using methods well known in the art.

Alternatively, an immunohistochemistry (IHC) method may be used. IHC specifically provides a method of detecting a target in a biological sample or tissue specimen in situ. The overall cellular integrity of the sample is maintained in IHC, thus allowing detection of both the presence and location of the target of interest. Typically a biological sample is fixed with formalin, embedded in paraffin and cut into sections for staining and subsequent inspection by light microscopy. Current methods of IHC use either direct labeling or secondary antibody-based or hapten-based labeling. Examples of known IHC systems include, for example, EnVision™ (DakoCytomation), Powervision® (Immunovision, Springdale, Ariz.), the NBA™ kit (Zymed Laboratories Inc., South San Francisco, Calif.), HistoFine® (Nichirei Corp, Tokyo, Japan).

In one embodiment of the present invention, direct sequencing of the whole genome is used to detect the SNP locus ABCA1. The whole genome sequencing may be achieved by use of the next generation sequencing (NGS) assay. In NGS, a single genomic DNA is first fragmented into a library of small segments that can be uniformly and accurately sequenced in millions of parallel reactions. The newly identified strings of bases, called reads, are then reassembled using a known reference genome as a scaffold (resequencing), or in the absence of a reference genome (de novo sequencing). The full set of aligned reads would reveal the entire sequence of each chromosome of the genomic DNA.

In another embodiment of the present invention, primer extension assay is used to detect the SNP locus ABCA1. The primer extension assay may be achieved by use of Matrix assisted laser desorption ionization time-of-flight mass spectrometry (MALDI-TOF MS). Mass spectrometry is an experimental technique used to identify the components of a heterogeneous collection of biomolecules, by sensitive discrimination of their molecular masses. In MALTI-TOF MS, the sample to be analyzed is placed in a UV-absorbing matrix pad and exposed to a short laser pulse. The ionized molecules are accelerated off the matrix pad (i.e., desorption) and move into an electric field towards a detector. The “time of flight” required to reach the detector depends on the mass/charge (m/z) ratio of the individual molecules. To use MALTI-TOF MS for DNA sequencing, the DNA sequence to be sampled is first transcribed into RNA in vitro in 4 separate reactions, each with three rNTP bases and one specific dNTP. The incorporated dNTP in the transcribed RNA will prevent cleavage from occurring at that dNTP position by RNAse, and therefore generate distinct fragments. Each fragment has a characteristic m/z ratio that appears as a peak in MALTI-TOF spectrum. The MALTI-TOF mass signal pattern obtained for the DNA sample is then compared with the expected m/z spectrum of the reference sequence, which includes the products of all 4 cleavage reactions. Any SNP differences between the sample DNA and the reference DNA sequences will produce predictable shifts in the spectrum, and their exact nature can be deduced.

In still another embodiment of the present invention, quantitative polymerase chain reaction (qPCR) is used to detect the desired SNP locus. In qPCR, DNA sample that includes the SNP locus is amplified and simultaneously detected and quantitated with different primer sets that target each allele separately. Well-designed primers will amplify their target SNP at a much earlier cycle than the other SNPs. This allows more than two alleles to be distinguished, although an individual qPCR reaction is required for each SNP. To achieve high enough specificity, the primer sequence may require placement of an artificial mismatch near its 3′-end, which is an approach generally known as Taq-MAMA. This artificial mismatch induces a much greater amplification delay for non-target alleles than a single mismatch would alone, yet does not substantially affect amplification of the target SNP.

In still another embodiment of the present invention, the SNP locus is detected by direct sequencing of a specified DNA segment containing the SNP locus of ABCA1.

As used herein, the term “expression level” refers to the expression level of ABCA1 with further other values corresponding to the clinical parameters. Typically, the expression level of the gene may be determined by any technology known by a person skilled in the art. In particular, each gene expression level may be measured at the genomic and/or nucleic and/or protein level. In a particular embodiment, the expression level of ABCA1 gene is measured. The expression level of ABCA1 is assessed by analyzing the expression of the protein translated from said gene. Said analysis can be assessed using an antibody (e.g., a radio-labelled, chromophore-labelled, fluorophore-labelled, or enzyme-labelled antibody), an antibody derivative (e.g., an antibody conjugate with a substrate or with the protein or ligand of a protein of a protein/ligand pair (e.g., biotin-streptavidin)), or an antibody fragment (e.g., a single-chain antibody, an isolated antibody hypervariable domain, etc.) which binds specifically to the protein translated from the gene encoding for ABCA1.

Methods for measuring the expression level of ABCA1 in a sample may be assessed by any of a wide variety of well-known methods from one of skill in the art for detecting expression of a protein including, but not limited to, direct methods like mass spectrometry-based quantification methods, protein microarray methods, enzyme immunoassay (EIA), radioimmunoassay (MA), Immunohistochemistry (IHC), Western blot analysis, ELISA, Luminex, ELISPOT and enzyme linked immunosorbent assay and indirect methods based on detecting expression of corresponding messenger ribonucleic acids (mRNAs). The mRNA expression profile may be determined by any technology known by a man skilled in the art. In particular, each mRNA expression level may be measured using any technology known by a man skilled in the art, including nucleic microarrays, quantitative Polymerase Chain Reaction (qPCR), next generation sequencing and hybridization with a labelled probe.

Said direct analysis can be assessed by contacting the sample with a binding partner capable of selectively interacting with the biomarker present in the sample. The binding partner may be an antibody that may be polyclonal or monoclonal, preferably monoclonal (e.g., a isotope-label, element-label, radio-labelled, chromophore-labelled, fluorophore-labelled, or enzyme-labelled antibody), an antibody derivative (e.g., an antibody conjugate with a substrate or with the protein or ligand of a protein of a protein/ligand pair (e.g., biotin-streptavidin)), or an antibody fragment (e.g., a single-chain antibody, an isolated antibody hypervariable domain, etc.) which binds specifically to the protein translated from the gene encoding for the biomarker of the invention. In another embodiment, the binding partner may be an aptamer.

The binding partners of the invention such as antibodies or aptamers, may be labelled with a detectable molecule or substance, such as an isotope, an element, a fluorescent molecule, a radioactive molecule or any others labels known in the art. Labels are known in the art that generally provide (either directly or indirectly) a signal.

As used herein, the term “labelled”, with regard to the antibody, is intended to encompass direct labelling of the antibody or aptamer by coupling (i.e., physically linking) a detectable substance, such as an isotope, an element, a radioactive agent or a fluorophore (e.g. fluorescein isothiocyanate (FITC) or phycoerythrin (PE) or Indocyanine (Cy5)) to the antibody or aptamer, as well as indirect labelling of the probe or antibody by reactivity with a detectable substance. An antibody or aptamer of the invention may be produced with a specific isotope or a radioactive molecule by any method known in the art. For example radioactive molecules include but are not limited to radioactive atom for scintigraphy studies such as 1123, 1124, In111, Re186, Re188, specific isotopes include but are not limited to 13C, 15N, 1261, 79Br, 81Br.

The aforementioned assays generally involve the binding of the binding partner (ie. antibody or aptamer) to a solid support. Solid supports which can be used in the practice of the invention include substrates such as nitrocellulose (e. g., in membrane or microtiter well form); polyvinylchloride (e. g., sheets or microtiter wells); polystyrene latex (e.g., beads or microtiter plates); polyvinylidene fluoride; diazotized paper; nylon membranes; activated beads, magnetically responsive beads, silicon wafers.

In a particular embodiment, an ELISA method can be used, wherein the wells of a microtiter plate are coated with a set of antibodies which recognize ABCA1 protein. A sample containing or suspected of containing said biomarker is then added to the coated wells. After a period of incubation sufficient to allow the formation of antibody-antigen complexes, the plate(s) can be washed to remove unbound moieties and a detectably labelled secondary binding molecule added. The secondary binding molecule is allowed to react with any captured sample marker protein, the plate washed and the presence of the secondary binding molecule detected using methods well known in the art such as Singulex, Quanterix, MSD, Bioscale, Cytof.

In one embodiment, an Enzyme-linked immunospot (ELISpot) method may be used. Typically, the sample is transferred to a plate which has been coated with the desired anti-ABCA1 protein capture antibodies. Revelation is carried out with biotinylated secondary Abs and standard colorimetric or fluorimetric detection methods such as streptavidin-alkaline phosphatase and NBT-BCIP and the spots counted.

In one embodiment, when multi-biomarker expression measurement is required, use of beads bearing binding partners of interest may be preferred. In a particular embodiment, the bead may be a cytometric bead for use in flow cytometry. Such beads may for example correspond to BD™ Cytometric Beads commercialized by BD Biosciences (San Jose, Calif.). Typically cytometric beads may be suitable for preparing a multiplexed bead assay. A multiplexed bead assay, such as, for example, the BD™ Cytometric Bead Array, is a series of spectrally discrete beads that can be used to capture and quantify soluble antigens. Typically, beads are labelled with one or more spectrally distinct fluorescent dyes, and detection is carried out using a multiplicity of photodetectors, one for each distinct dye to be detected. A number of methods of making and using sets of distinguishable beads have been described in the literature. These include beads distinguishable by size, wherein each size bead is coated with a different target-specific antibody (see e.g. Fulwyler and McHugh, 1990, Methods in Cell Biology 33:613-629), beads with two or more fluorescent dyes at varying concentrations, wherein the beads are identified by the levels of fluorescence dyes (see e.g. European Patent No. 0 126,450), and beads distinguishably labelled with two different dyes, wherein the beads are identified by separately measuring the fluorescence intensity of each of the dyes (see e.g. U.S. Pat. Nos. 4,499,052 and 4,717,655). Both one-dimensional and two-dimensional arrays for the simultaneous analysis of multiple antigens by flow cytometry are available commercially. Examples of one-dimensional arrays of singly dyed beads distinguishable by the level of fluorescence intensity include the BD™ Cytometric Bead Array (CBA) (BD Biosciences, San Jose, Calif.) and Cyto-Plex™ Flow Cytometry microspheres (Duke Scientific, Palo Alto, Calif.). An example of a two-dimensional array of beads distinguishable by a combination of fluorescence intensity (five levels) and size (two sizes) is the QuantumPlex™ microspheres (Bangs Laboratories, Fisher, Ind.). An example of a two-dimensional array of doubly-dyed beads distinguishable by the levels of fluorescence of each of the two dyes is described in Fulton et al. (1997, Clinical Chemistry 43(9):1749-1756). The beads may be labelled with any fluorescent compound known in the art such as e.g. FITC (FL1), PE (FL2), fluorophores for use in the blue laser (e.g. PerCP, PE-Cy7, PE-Cy5, FL3 and APC or Cy5, FL4), fluorophores for use in the red, violet or UV laser (e.g. Pacific blue, pacific orange). In another particular embodiment, bead is a magnetic bead for use in magnetic separation. Magnetic beads are known to those of skill in the art. Typically, the magnetic bead is preferably made of a magnetic material selected from the group consisting of metals (e.g. ferrum, cobalt and nickel), an alloy thereof and an oxide thereof. In another particular embodiment, bead is bead that is dyed and magnetized.

In one embodiment, protein microarray methods may be used. Typically, at least one antibody or aptamer directed against ABCA1 protein is immobilized or grafted to an array(s), a solid or semi-solid surface(s). A sample containing or suspected of containing ABCA1 protein is then labelled with at least one isotope or one element or one fluorophore or one colorimetric tag that are not naturally contained in the tested sample. After a period of incubation of said sample with the array sufficient to allow the formation of antibody-antigen complexes, the array is then washed and dried. After all, quantifying ABCA1 protein may be achieved by using any appropriate microarray scanner like fluorescence scanner, colorimetric scanner, SIMS (secondary ions mass spectrometry) scanner, maldi scanner, electromagnetic scanner or any technique allowing to quantify said labels.

In another embodiment, the antibody or aptamer grafted on the array is labelled.

In another embodiment, reverse phase arrays may be used. Typically, at least one sample is immobilized or grafted to an array(s), a solid or semi-solid surface(s). An antibody or aptamer against the suspected biomarker is then labelled with at least one isotope or one element or one fluorophore or one colorimetric tag that are not naturally contained in the tested sample. After a period of incubation of said antibody or aptamer with the array sufficient to allow the formation of antibody-antigen complexes, the array is then washed and dried. After all, detecting quantifying and counting by D-SIMS said biomarker containing said isotope or group of isotopes, and a reference natural element, and then calculating the isotopic ratio between the biomarker and the reference natural element. may be achieve using any appropriate microarray scanner like fluorescence scanner, colorimetric scanner, SIMS (secondary ions mass spectrometry) scanner, maldi scanner, electromagnetic scanner or any technique allowing to quantify said labels.

In one embodiment, said direct analysis can also be assessed by mass Spectrometry. Mass spectrometry-based quantification methods may be performed using either labelled or unlabelled approaches (DeSouza and Siu, 2012). Mass spectrometry-based quantification methods may be performed using chemical labeling, metabolic labelingor proteolytic labeling. Mass spectrometry-based quantification methods may be performed using mass spectrometry label free quantification, LTQ Orbitrap Velos, LTQ-MS/MS, a quantification based on extracted ion chromatogram EIC (progenesis LC-MS, Liquid chromatography-mass spectrometry) and then profile alignment to determine differential expression of the biomarker.

In another embodiment, the ABCA1 expression level is assessed by analyzing the expression of mRNA transcript or mRNA precursors, such as nascent RNA, of ABCA1 gene. Said analysis can be assessed by preparing mRNA/cDNA from cells in a sample from a subject, and hybridizing the mRNA/cDNA with a reference polynucleotide. The prepared mRNA/cDNA can be used in hybridization or amplification assays that include, but are not limited to, Southern or Northern analyses, polymerase chain reaction analyses, such as quantitative PCR (TaqMan), and probes arrays such as GeneChip™ DNA Arrays (AFFYMETRIX).

Advantageously, the analysis of the expression level of mRNA transcribed from the gene encoding for biomarkers involves the process of nucleic acid amplification, e. g., by RT-PCR (the experimental embodiment set forth in U.S. Pat. No. 4,683,202), ligase chain reaction (Barany, 1991), self-sustained sequence replication (Guatelli et al., 1990), transcriptional amplification system (Kwoh et al., 1989), Q-Beta Replicase (Lizardi et al., 1988), rolling circle replication (U.S. Pat. No. 5,854, 033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

As used herein, the term “predetermined reference value” refers to a threshold value or a cut-off value. The setting of a single “reference value” thus allows discrimination between a subject at risk of having or developing IA and a subject not at risk of having or developing IA with respect to the overall survival (OS) for a subject. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. Preferably, the person skilled in the art may compare the expression level (obtained according to the method of the invention) with a defined threshold value. In one embodiment of the present invention, the threshold value is derived from the expression level (or ratio, or score) determined in a biological sample derived from one or more subjects at risk of having or developing CMML. Furthermore, retrospective measurement of the expression level (or ratio, or scores) in properly banked historical subject samples may be used in establishing these threshold values.

Predetermined reference values used for comparison may comprise “cut-off” or “threshold” values that may be determined as described herein. Each reference (“cut-off”) value for ABCA1 may be predetermined by carrying out a method comprising the steps of

a) providing a collection of samples from subjects at risk of having or developing CMML;

b) determining the expression level of ABCA1 protein for each sample contained in the collection provided at step a);

c) ranking the biological samples according to said expression level;

d) classifying said samples in pairs of subsets of increasing, respectively decreasing, number of members ranked according to their expression level,

e) providing, for each sample provided at step a), information relating to the risk of having or developing CMML or the actual clinical outcome for the corresponding subject (i.e. the duration of the overall survival (OS));

f) for each pair of subsets of samples, obtaining a Kaplan Meier percentage of survival curve;

g) for each pair of subsets of samples calculating the statistical significance (p value) between both subsets;

h) selecting as reference value for the expression level, the value of expression level for which the p value is the smallest.

For example the expression level of ABCA1 has been assessed for 100 samples of 100 patients. The 100 samples are ranked according to their expression level. Sample 1 has the best expression level and sample 100 has the worst expression level. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding patient, Kaplan Meier curves are prepared for each of the 99 groups of two subsets. Also for each of the 99 groups, the p value between both subsets was calculated.

The reference value is selected such as the discrimination based on the criterion of the minimum p value is the strongest. In other terms, the expression level corresponding to the boundary between both subsets for which the p value is minimum is considered as the reference value. It should be noted that the reference value is not necessarily the median value of expression levels.

In routine work, the reference value (cut-off value) may be used in the present method to discriminate samples and therefore the corresponding patients.

Kaplan-Meier curves of percentage of survival as a function of time are commonly to measure the fraction of patients living for a certain amount of time after treatment and are well known by the man skilled in the art.

The man skilled in the art also understands that the same technique of assessment of the expression level of ABCA1 should of course be used for obtaining the reference value and thereafter for assessment of the expression level of a biomarker of a patient subjected to the method of the invention.

In one embodiment, the reference value may correspond to the expression level of ABCA1 determined in a sample associated with subject at risk of having or developing CMML. Accordingly, a lower expression level of ABCA1 than the reference value is indicative of a subject at risk of having or developing CMML, and a higher or equal expression level of ABCA1 than the reference value is indicative of a subject not at risk of having or developing CMML.

In another embodiment, the reference value may correspond to the expression level of ABCA1 determined in a sample associated with subject not at risk of having or developing CMML. Accordingly, a higher or equal expression level of ABCA1 than the reference value is indicative of a subject not at risk of having or developing CMML, and a lower expression level of ABCA1 than the reference value is indicative of a subject at risk of having or developing CMML.

Method for Treating Chronic Myelomonocytic Leukemia

In a third aspect, the invention relates to a method for treating CMML in a subject in need thereof comprising a step of administering to said subject a therapeutically effective amount of: HDL/ABCA recombinant (ApoA-1); cylodextrin and/or anti-IL-3Rbeta antibody.

As used herein, the terms “treating” or “treatment” refer to both prophylactic or preventive treatment as well as curative or disease modifying treatment, including treatment of subject at risk of contracting the disease (CMML) or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

In a particular embodiment, the invention relates to a method for treating CMML in a subject in need thereof comprising a step of administering to said subject a therapeutically effective amount of HDL.

As used herein, the term “HDL” refers to high-density lipoprotein. It is the smallest of the lipoprotein particles. It is the densest because it contains the highest proportion of protein to lipids. Its most abundant apolipoproteins are apo A-I and apo A-II. HDL transports cholesterol mostly to the liver or steroidogenic organs such as adrenals, ovary, and testes by both direct and indirect pathways.

In a further embodiment, the a method for treating CMML in a subject in need thereof comprising a step of administering to said subject a therapeutically effective amount of ABCA 1 recombinant (ApoA-1).

As used herein, the term “ApoA-1” also known as ETC-216, MDCO-216 is a naturally occurring mutated variant of the apolipoprotein Al protein found in human HDL.

In a particular embodiment, the invention relates to a method for treating CMML in a subject in need thereof comprising a step of administering to said subject a therapeutically effective amount of cylodextrin.

As used herein, the term “cylodextrin” belongs to a family of cyclic oligosaccharides, consisting of a macrocyclic ring of glucose subunits joined by α-1,4 glycosidic bonds.

In a particular embodiment, the invention relates to a method for treating CMML in a subject in need thereof comprising a step of administering to said subject a therapeutically effective amount of anti-IL-3Rbeta antibody.

As used herein, the term “IL-3 R beta” refers to a molecule found on cells which helps transmit the signal of interleukin-3, a soluble cytokine important in the immune system. In the context of the invention, the anti-IL-3 R beta antibody is a blocking antibody prevented bone marrow proliferation.

In a particular embodiment, the anti-IL-3Rbeta antibody is selected from the group but not limited to: MAB5491, AF549 (FAB5492A) or GWB-ASC 152.

In a further embodiment, the method according to the invention, wherein cylodextrin and anti-IL-3Rbeta antibody are administered simultaneously separately or sequentially as a combined preparation.

As used herein the terms “administering” or “administration” refer to the act of injecting or otherwise physically delivering a substance as it exists outside the body (e.g., HDL/ABCA recombinant (ApoA-1); cylodextrin and/or anti-IL-3Rbeta antibody) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

By a “therapeutically effective amount” is meant a sufficient amount of HDL/ABCA recombinant (ApoA-1); cylodextrin and/or anti-IL-3Rbeta antibody for use in a method for treating CMML in a subject in need thereof at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic 20 adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Kit

In a fourth aspect, the invention relates to a kit for performing the methods of the present invention, wherein said kit comprises means for measuring at least one mutation as described above in ABCA1 protein and/or detecting ABCA1 SNP that is indicative of the risk of having a short survival time in a subject.

Typically the kit may include antibodies, primers, probes, macroarrays or microarrays as above described. For example, the kit may comprise a set of antibodies, primers, or probes as above defined, and optionally pre-labelled. Alternatively, antibodies, primers, or probes may be unlabelled and the ingredients for labelling may be included in the kit in separate containers.

The kit may further comprise hybridization reagents or other suitably packaged reagents and materials needed for the particular hybridization protocol, including solid-phase matrices, if applicable, and standards. The kit may further comprise amplification reagents and also other suitably packaged reagents and materials needed for the particular amplification protocol.

The invention will be further illustrated by the following figures and examples. However, these examples and figures should not be interpreted in any way as limiting the scope of the present invention.

### FIGURES

FIG. 1. Identification of loss of function ABCA1 mutants in CMML. (A) [3H]-thymidine proliferation assay (pulsed for 2 h) were performed in HEK293 cells transiently transfected with plasmid constructs expressing ABCA1-WT, ABCA1 mutants or empty vector. (B) Abca1 transcripts expressed as arbitrary unit (a.u.) in several leukemic cell lines. THP-1 cells are monocytic leukemia cells that express relatively high amount of Abca1 transcripts compared to myeloblastic MV411 and HL60 cells, acute myeloid leukemia (AML) HEL, OCIAML3, MOLM13 and KG1 cells, chronic myeloid leukemia (CML) MOLM6 and K562 cells or lymphoma U237 and OCILY3 cells (C) or in THP-1 monocytic leukemia cells transduced for 72 h with lentiviral particles expressing ABCA1-WT, ABCA1 mutants or empty vector. (D) THP-1 cells transduced for 72 h with ABCA1 mutants exhibit growth advantage over a 7-day period compared with ABCA1-WT. (E) Expression of phosphoSTAT5. (F) Cholesterol efflux from the aforementioned THP-1 macrophages was quantified after overnight [³H]-cholesterol loading and determined 6 hours after incubation with apoA-I (15 μg/mL) and BODIPY staining (G) determined by flow cytometry in these cells.

FIG. 2. Loss of functional ABCA1 reduces tumor suppression in myelomonocytic leukemia-induced by Tet2 loss. (A) Modulation of Abca1 mRNA expression levels in the BM of the aforementioned mouse models. (B) Quantification of the percentage of peripheral blood myeloid cells determined by hematology cell counter over the course of 12 weeks after Poly:IC injection in recipients mice transplanted with empty, ABCA1-WT or ABCA1 mutants expressing Mx1-Cre⁺Tet2^{fl/fl} BM. (C) Peripheral blood myeloid subsets (CD115⁺Ly6C^{hi} and CD115⁺Ly6C^{lo} monocytes and CD115⁻Ly6C^{hi} neutrophils) were also quantified in these mice at the indicated time point. (D) Original magnification, ×200. Arrows indicate extensive cellular infiltrate. Quantification of spleen weight of these mice. (E) Representative quantification of CD11b⁺ GrI⁺ myeloid cells determined by flow cytometry in the spleen of recipient mice transplanted with control or Mx1-Cre⁺Tet2^{fl/fl} BM expressing empty, ABCA1-WT or ABCA1 mutants. The results are means ±SEM of 5-9 animals per group. ND, not detectable. * P<0.05 versus empty control on a Tet2 deficient background. § P<0.05 versus ABCA1-WT #P<0.05 and ##P<0.001 versus Mx1-Cre⁺ controls.

FIG. 3. ABCA1 mutants supports Tet2-deficient HSPC expansion and myeloid lineage commitment and spreads CMML-like disease in serial BM transplantation. Quantification of hematopoietic stem (A) and progenitor (B) cells in the BM of recipient mice transplanted with control or Mx1-Cre⁺ Tet2^{fl/fl} BM expressing empty, ABCA1-WT or ABCA1 mutants. Lineage(Lin)⁻ Sca1⁻c-Kit⁺ LSK cells are hematopoietic stem and progenitor cells; Lin⁻Sca1⁻c-Kit⁺CD34^{hi}FcγR^{hi} GMPs are granulocyte-monocyte progenitors and Lin⁻Sca1⁻c-Kit⁺CD34^{hi}FcγR^{low} CMPs are common myeloid progenitors. Results are mean±SEM of 5-9 animals per group. (C) Quantification of hematopoietic progenitors and (D) myeloid cells in BM cultures isolated from Mx1-Cre⁺Tet2^{fl/fl} BM expressing empty, ABCA1-WT or ABCA1 mutants and grown ex vivo for 72 h in liquid culture in presence or absence of 6 ng/ml IL-3 and 2 ng/mL GM-CSF. (E) Spleen weight or (F) cholesterol content of recipient mice serially transplanted with control or Mx1-Cre⁺Tet2^{fl/fl} BM expressing empty, ABCA1-WT or ABCA1 mutants. Results are means±SEM of 5-9 animals per group. * P<0.05 versus empty control on a Tet2 deficient background. § P<0.05 versus ABCA1-WT #P<0.05 versus Mx1-Cre⁺ controls.

FIG. 4. ABCA1 invalidation propagates myelopoiesis and accelerates extramedullary hematopoiesis on a Tet2 deficient background. (A) Modulation of Abca1 and Tet2 mRNA expression levels in the BM of the aforementioned mouse models. (B) Quantification of the percentage of peripheral blood myeloid cells determined by hematology cell counter over the course of 20 weeks after Poly:IC injection in recipients mice transplanted with the BM from Mx1-Cre⁺, Mx1-Cre⁺Abca1^{fl/fl}, MX1-Cre⁺Tet2^{fl/fl} and Mx1-Cre⁺Tet2^{fl/fl}Abca1^{fl/fl} mice. (C) Peripheral blood myeloid subsets (CD115⁺Ly6C^{hi} and CD115⁺Ly6C^{lo} monocytes and CD115⁻Ly6C^{hi} neutrophils) were also quantified in these mice at the indicated time point. (D) Original magnification, ×200. Arrows indicate extensive cellular infiltrate. Quantification of myeloid subsets (oesinophils, neutrophils, monocytes and red pulp macrophages (RPMs)) in the spleen. (E) Quantification of hematopoietic stem and (F, G) progenitor cells in the BM of these mice. Lineage(Lin)⁻Sca1⁻c-Kit⁺ LSK cells are hematopoietic stem and progenitor cells; Lin⁻Sca1⁻cKit⁺CD34^{hi}FcγR^{low} MEPs are megakaryocyte-erythrocyte progenitors; Lin⁻Sca1⁻c-Kit⁺CD34^{hi}FcγR^{hi} GMPs are granulocyte-monocyte progenitors and Lin⁻Sca1⁻c-Kit⁺CD34^{hi}FcγR^{low} CMPs are common myeloid progenitors. Results are mean±SEM of 5-7 animals per group. ND, not detectable. #P<0.05 and ##P<0.001 versus Mx1-Cre⁺ controls.

FIG. 5. Cholesterol accumulation couples ABCA1 invalidation and Tet2 deficiency to IL-3 receptor β signaling hypersensitivity. (A-D) Quantification of BODIPY staining by flow cytometry expressed as mean fluorescence intensity (MFI) as a surrogate of cellular cholesterol neutral lipid per cell in BM hematopoietic stem (A and C) and progenitor (B and D) cells (i.e, LSKs, MEPs, CMPs and GMPs) of recipient mice transplanted with Mx1-Cre⁺, Mx1-Cre⁺ Abca1^{fl/fl}, MX1-Cre⁺Tet2^{fl/fl} and Mx1-Cre⁺Tet2^{fl/fl}Abca1^{fl/fl} BM (A and B) or control and Mx1-Cre⁺Tet2^{fl/fl} BM expressing empty, ABCA1-WT or ABCA1 mutants (C and D). (E) Expression of mRNA was normalized to m36B4. mRNA levels were expressed as percentage over WT whole BM cells. Proliferation rates were determined after 2 h [³H]-thymidine pulse labeling in BM cells from empty, ABCA1-WT and ABCA1 mutants-transduced animals on a Tet2 deficient background that were grown for 72 h in liquid culture in the presence or absence of 6 ng/mL IL-3 and 2 ng/mL GM-CSF and the indicated chemical compounds.

FIG. 6. HDL overcome loss-of-function ABCA1 mutants and limit the myeloproliferative disorder induced by these mutants in Tet2 deficient mice. (A) Proliferation rates were determined after 2 h [³H]-thymidine pulse labeling in BM cells from empty, ABCA1-WT and ABCA1 mutants-transduced animals on a Tet2 deficient background that were grown for 72 h in liquid culture in the presence or absence of 50 μg/mL PEG-HDL. (B) Quantification of BODIPY staining was determined by flow cytometry in these cells and expressed as the mean fluorescence intensity (MFI). (C) Results are means±SEM of cultures from at least three independent mice. (E) Levels of hapoA-I (D) and plasma HDL-cholesterol levels were determined in WT or apoA-I transgenic recipient mice transplanted with Mx1-Cre⁺Tet2^{fl/fl} BM transduced with lentiviral particles expressing ABCA1-WT, ABCA1 mutants or empty vector. (F) Peripheral blood CD11b⁺GrI⁺ myeloid subsets and spleen weight were quantified at the end of the study period (i.e, 7 weeks post Poly:IC injection that followed a 5 week recovery period post BMT) in WT or apoA-I transgenic recipient mice transplanted with Mx1-Cre⁺Tet2^{fl/fl}BM transduced with lentiviral particles expressing ABCA1-WT, ABCA/mutants or empty vector. Results are means±SEM of 4-5 animals per group. * P<0.05 versus empty control transduced animals on a Tet2 deficient background. § P<0.05 versus ABCA1-WT #P<0.05 versus non-HDL treated conditions or transduced animals on a non-ApoAI^{Tg} background.

### EXAMPLE

### Material & Methods

Genetic Analysis of Primary Patient Samples.

Peripheral blood and/or bone marrow samples were collected from 26 patients with CMML; informed consent was obtained from all patients included in this study. Matched normal tissue in the form of a buccal swab was available for all patients. Genomic DNA was extracted from viably frozen peripheral blood granulocytes and buccal swabs. High-throughput DNA sequence analysis was used to screen for mutations in ABCA1, ABCG1, NR1H2, and NR1H3. All DNA samples were whole genome amplified using ∅29 polymerase to ensure sufficient material was available for sequence analysis. M13-appended gene-specific primers were designed to amplify and sequence all coding exons of all isoforms of the above mentioned genes. Primer sequences and the genomic coordinates of all amplicons sequenced are included in Supplemental Table 1. Bidirectional sequence traces were analyzed for missense and nonsense mutations using Mutation Surveyor (Softgenetics, Inc., State College, Pa.), and all traces were reviewed manually and with Mutation Surveyor for the presence of frameshift mutations. Mutations were annotated according to the predicted effects on coding sequence using NM_005502.2, NM_004915.3, NM_007121.4, and NM_001130101.1 as the reference sequence for ABCA1, ABCG1, NR1H2, and NR1H3 respectively. Non-synonymous mutations were first compared to published SNP data (dbSNP, http://www.ncbi.nlm.nih.gov/projects/SNP) such that previously annotated SNPs were not considered pathogenic mutations. Missense mutations not in the published SNP database were annotated as somatic mutations based on either on reported data demonstrating these are somatic mutations or sequence analysis of that demonstrated these mutations were present in tumor and not in matched normal DNA. All somatic mutations were validated by resequencing non-amplified source DNA for the particular amplicon where the mutation was noted. Genomic DNA from paired samples was verified to belong to the same patient by genotyping of the specimens for 42 highly polymorphic single-nucleotide polymorphisms using mass-spectrometry based genotyping as described previously. In order to determine whether the genes in which non-synonymous mutations were identified were mutated at a rate higher than expected by chance alone, we first calculated the rate of non-synonymous mutations in the sequenced genes. We then performed binomial test in R (http://www.r-project.org/) to compare the rate of non-synonmous mutations in the genes identified in this study with the expected background rate of 0.22-2.5×10⁻⁶ synonymous mutations identified in several prior large-scale sequencing studies²⁻¹⁰ as well as the expected ratio of silent:non-silent mutations (0.31-0.41) from the same studies.

Mice and Treatments.

WT, Mx1-Cre⁺ (B6. Cg-Tg(Mx1-cre)^{1CgnJ}), Tet2^{fl/fl} (B6; 129 S-Tet2^{tm1.1Iaai/J}) Abca1^{fl/fl} mice (B6. 129S6-Abca1^{tm1IJp/J}) and human apoA-1 transgenic (B6.Tg(ApoA1)^{1Rub/J}), were obtained from the Jackson Laboratory. Human apoA-1 transgenic mice were selected based on the human apoA-1 levels in the range of 150-300 mg/dL (ELISA do not detect mouse apoA-1) as previously described (Rubin et al., 1991; Yvan-Charvet et al., 2010). Mx1-Cre⁺ Tet2^{fl/fl} mice, Mx1-Cre⁺ Abca1^{fl/fl} mice and Mx1-Cre⁺ Tet2^{fl/fl}Abca1^{fl/fl} littermates mice were used for this study. Bone marrow (BM) transplantation into lethally irradiated WT recipients and serial BM transplantation studies were performed as previously described ((Yvan-Charvet et al., 2010)). After 5 weeks of reconstitution, mice were i.p injected with poly:IC (250 μg/injection with a cumulative dose of 750 μg/mice, Invivogen) to induce gene deletion/recombination. Mice were used between 3 and 5 months after the injections of poly:IC depending of the experiment.

Animal procedures were conducted in accordance with the Guidelines for the Care and Use of Laboratory Animals and were approved by the Institutional Animal Care and Use Committees at Mediterranean Center of Molecular Medicine (C3M). Mice were maintained on a 12 h light/12 h darkness lighting schedule. Animals had ad libitum access to both food and water.

Plasmids.

Mouse ABCA1 cDNA, with a homology of 97% to human ABCA1 cDNA, was used to generate P711L, A1291T, G1421R, P1423S and A2011T mutant cDNAs and cloned into pLKO lentiviral vectors to genetically perturb cells by lentiviral infection and avoid cross reactivity.

Lentiviral BM Transplantation.

The lentiviral BM transplant assay was performed as previously described (Gautier et al., 2013). In brief, Mx1-Cre⁺ and Mx1-Cre⁺ Tet2^{fl/fl} mice were injected with 5-fluorouracile (3 mg/mice of 5-FU, F6627, Sigma) 3 days before the experiment to enrich HSPCs within the BM. Control, ABCA1-WT and ABCA1-mutant lentiviral particles (pLKO lentiviral vector containing a MSCV-IRES-EGFP sequence, Genecust) were tittered and used to transduce Mx1-Cre⁺ or Mx1-Cre⁺ Tet2^{fl/fl} cells. BM cells were cultured for 24 h in transplantation media (RPMI+10% FBS+6 ng/ml IL-3 (Corning), 10 ng/ml IL-6, and 10 ng/ml stem cell factor (Milteny Biotech)) and treated with lentiviral particles (MOI of 5 in the presence of polybrene (Sigma)). After washing, the cells were used for BM transplantation into lethally irradiated WT or human apoA-1 transgenic recipient mice as indicated in the figure legends. The transduction efficiency ranged from 70-90% in LSK cells before implantation as previously described (Gough et al., 2003) (Pikman et al., 2006) (Westerterp et al., 2012). After 5 weeks of reconstitution, mice were i.p injected with poly:IC (250 μg/injection with a cumulative dose of 750 μg/mice, Invivogen) to induce gene deletion/recombination. Mice were used between 3 and 5 months after the injections of poly:IC depending of the experiment.

White Blood Cell Counts

Leukocytes, differential blood counts, platelets and erythrocytes were quantified from whole blood using a hematology cell counter (HEMAVET® 950).

Histopathology

Mice were euthanized and tissues were harvested and fixed in 4% paraformaldehyde. Spleen was serially paraffin sectioned using a Microm HM340E microtome (Microm Microtech, Francheville France) and stained with H&E for morphological analysis as previously described (Yvan-Charvet et al., 2010).

HEK293 cell transfection and culture. HEK293 cells (human embryonic kidney, CRL-1573, ATCC) at a density of 10⁶ cells/well were transiently transfected with similar amounts of control empty vector (pcDNA 3.1⁺), ABCA1-WT or mutant cDNA using LipofectAMINE 2000 according to the manufacturer's instructions (Invitrogen). Then, cells were incubated for different times in DMEM containing 10% FBS before treatments as indicated in the figure legends.

Human THP-1 monocytic leukemia cells and treatments. THP-1 monocytes (human acute monocytic leukemia cell line, TIB-202, ATCC) were cultured in RPMI 1640 medium supplemented with 10% fetal bovine serum (FBS) at 37° C. in 5% CO2. Non-adherent monocytes were transduced at MOI of 5 with control, ABCA1-WT and ABCA1-mutant lentiviral particles (pLKO lentiviral vector containing a CMV promoter, Genecust) in the presence of polybrene (Sigma) and used 3 days later for experiments as described in the figure legends. THP1 cells were treated for 16 hours with puromycin 24 hours after transfection to improve the transient transduction/transfection efficiency up to 60% to 80% (data not shown), which slowed down the proliferation rate of these cells. In some experiments, stable overexpressing ABCA1-WT and ABCA/-mutants THP-1 macrophages were generated after lentiviral transduction and GFP selection of a puromycin resistant pLKO vector containing ABCA/gene.

BM Harvest and Treatment

Briefly, femurs were flushed with ice-cold PBS and centrifugated for 5 min at 1,000 rpm to extract BM cells. After red blood cell lysis, over 90% of BM cells were CD45-positive cells of hematopoietic origin (Westerterp et al., 2012). Primary BM cells were resuspended in IMDM (Gibco) containing 10% FCS (STEMCELL Technologies) and cultured for 1 h in tissue culture flasks to remove adherent cells, including macrophages. The transduction rate of control, ABCA1-WT and ABCA 1-mutant lentiviral particles was determined after BM transplantation as described above. Suspended cells were then normalized to the same concentration and cultured for 72 h in the presence of 6 ng/mL IL-3 and 2 ng/mL GM-CSF (R&D Systems). In some experiments, the cyclodextrin (Sigma) was used at the final concentration of 5 mM, tempol (EMD Millipore) at 4 mM and anti-IL3Rbeta AF549 antibody (R&D Systems) at 50 μg/mL.

[³H]-Thymidine Proliferation Assay

For proliferation assays, cells were pulsed for 2 h with 2 μCi/ml [³H]-thymidine, and the radioactivity incorporated into the cells was determined by standard procedures using a liquid scintillation counter.

Isotopic Cholesterol Efflux Assay

THP-1 monocytes were treated with 100 nmol/L PMA (Phorbol myristate acetate) for 24 hour to facilitate differentiation into macrophages and cultured for 24 h in RPMI 1640 medium supplemented with 10% fetal bovine serum (FBS) containing 2 μCi/ml of [3H]-cholesterol. Cholesterol efflux was performed for 6 h in 0.2% BSA DMEM containing 15 μg/mL apoA-I. The cholesterol efflux was expressed as the percentage of the radioactivity released from the cells in the medium relative to the total radioactivity in cells plus medium (Yvan-Charvet et al., 2010).

Cellular and Tissue Cholesterol Content

Total lipids were extracted with chloroform/methanol from total cell lysates. Cholesterol mass in cells was determined using colorimetric kits (Wako Chemicals).

Flow Cytometry Analysis.

BM cells, peripheral blood and splenocytes were collected from leg bones, blood and spleen cells after manual flushing or grinding, lysis to remove red blood cells and filtering through a 40-μm cell strainer as previously described (Yvan-Charvet et al., 2010). For peripheral blood leukocytes analysis, 100 μL of blood were collected into EDTA tubes before red blood cell lysis and filtration. Freshly isolated BM, spleen and blood cells were stained with the appropriate antibodies for 30 min on ice. Cellular cholesterol content was quantified using the Bodipy-cholesterol probe (Life Technologies). Phosphoflow staining was performed according to the manufacturer's instruction (BD Biosciences). HSPC and hematopoietic progenitor subsets and myeloid cell populations were analyzed by flow cytometry using an LSR Fortessa (Becton Dickinson) or sorted with a FACSAria II instrument (Becton Dickinson). All gating strategies are depicted in the Figures. Data were analyzed with FlowJo software (Tree Star).

Cholera Toxin staining

After a wash in complete growth medium, THP-1 transduced monocytes were stained 15 min at 37° C., in 1 μg/ml working solution of Cholera Toxin Subunit B, Alexa Fluor 594 conjugate (Invitrogen, C34777). Cells were then stained with 1 ng/ml working solution of DAPI (4′,6-diamidino-2-phenylindole) and washed 3 times in PBS1X. Immunostaining of cells was read on a Nikon Confocal MR microscope.

Antibodies. TCR-β (H57-597), F4/80 (BM8), CD2 (RM2-5), CD3e (145-2C11), CD4 (GK1.5), CD8b (53-6.7), CD19 (eBio1D3), CD45R (B220, RA3-6B2), Gr-1 (Ly6G, RB6-8C5), Cd11b (Mac1, M1/70), Ter119 (Ly76) and NK1.1 (Ly53, PK136)-FITC were all from eBioscience and used for lineage determination. c-Kit (CD117, ACK2)-APCeFluor780 from eBioscience, Sca-1-Pacific blue from Biolegend, FcgRII/III-PE (CD16/32, 2.4G2), CD34 (RAM34)-AlexaFluor 647, CD135 (Flt3, A2F10)-PE, CD150 (Slamf1, TC15-12F 12.2)-PECy7 were from Biolegend and used to quantify HSPCs and progenitor subsets. Peripheral leukocytes were stained with CD115 (AFS98)-APC, CD45 (30-F11)-APCCy7 and Ly6C/G or Gr-1 (RB6-8C5)-PercPCy5.5 from eBioscience and BD Biosciences, respectively.

RNA analysis

Total RNA extraction, cDNA synthesis and real-time PCR were performed as described previously (16). m36B4 RNA expression was used to account for variability in the initial quantities of mRNA.

Western Blotting

The expression of ABCA1, TET2, phospho JAK2 and ERK were measured in BM cell by Western blot analysis. Briefly, cell extracts were electrophoresed on 4-20% gradient SDS-PAGE gels and transferred to 0.22-μm nitrocellulose membranes. The membrane was blocked in Tris-buffered saline, 0.1% Tween20 containing 5%(w/v) nonfat milk (TBST-nfm) at room temperature (RT) for 1 h and then incubated with the primary antibody (all from Cell Signaling) in TBST-nfm at RT for 4 h, followed by incubation with the appropriate secondary antibody coupled to horseradish peroxidase. Proteins were detected by ECL chemiluminescence (Pierce). Intensity of each protein strips was quantified using Image J software.

Statistical Analysis

Data are shown as mean±SEM. Statistical significance was performed using two-tailed parametric student's t test or by one-way analysis of variance (ANOVA, 4-group comparisons) with a Bonferroni multiple comparison post-test according to the dataset (GraphPad software, San Diego, Calif.). Results were considered as statistically significant when P<0.05.

Results

Identification of ABCA1 Somatic Mutations in CMML

Sequencing of full-length ABCA1, ABCG1 and NR1H2/3 (LXRs) in 26 CMML samples revealed a somatic mutational frequency of 19% of samples for ABCA1 (n=5) and 0% for ABCG1 and NR1H2/3. All mutations were somatic missense mutations with only one mutation observed in each patient sample (data not shown). The identity of the paired samples was verified by Sequenom SNP genotyping demonstrating that the likelihood of a match occurring by chance was <1×10-13 (data not shown). These ABCA1 mutations occur in evolutionarily conserved regions (data not shown). The ABCA1 mutations have not been previously described even though different ABCA1 mutations have been identified in Tangier Disease (Brunham et al., 2006; Sjöblom et al., 2006). Sequencing of other genes implicated in the pathogenesis of CMML in these same samples revealed that ABCA1 mutations co-existed with known oncogenic mutations in JAK2, Flt3, and N-Ras (Emanuel, 2008). We noted that (1) of the 4 genes sequenced, somatic non-synonymous mutations were found in only 2 of the 4 genes and (2) the somatic nonsynonymous mutation rate for ABCA1 was higher than the expected background silent mutation rate and higher than expected by chance alone by binomial tests (p-value of 3.6×10-10 for ABCA1), suggesting that mutations in ABCA1 do not represent passenger gene effects.

Functional Analysis of ABCA1 Mutations In Vitro

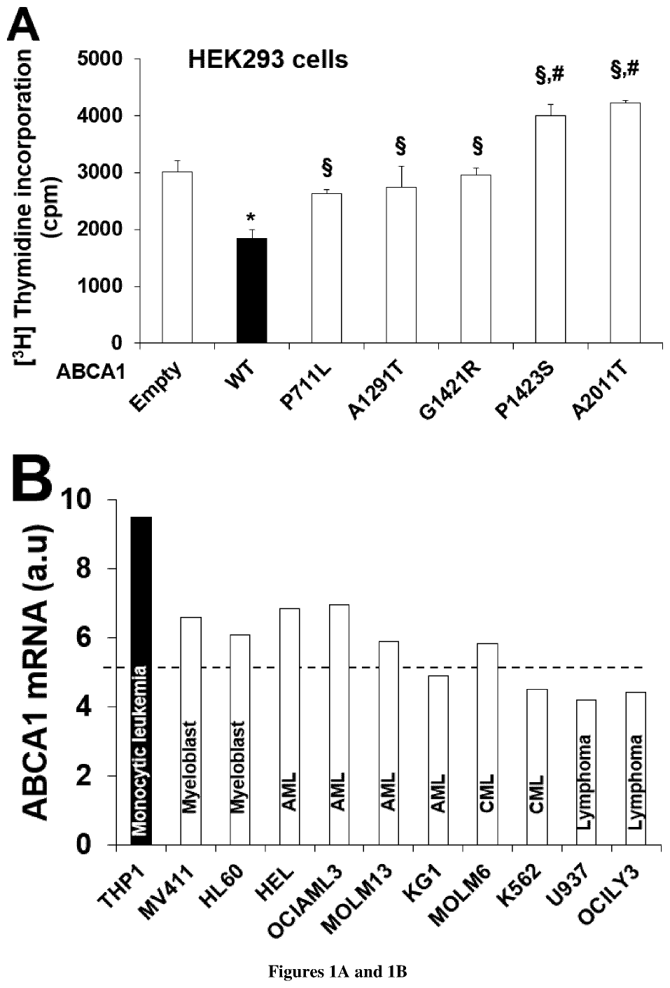

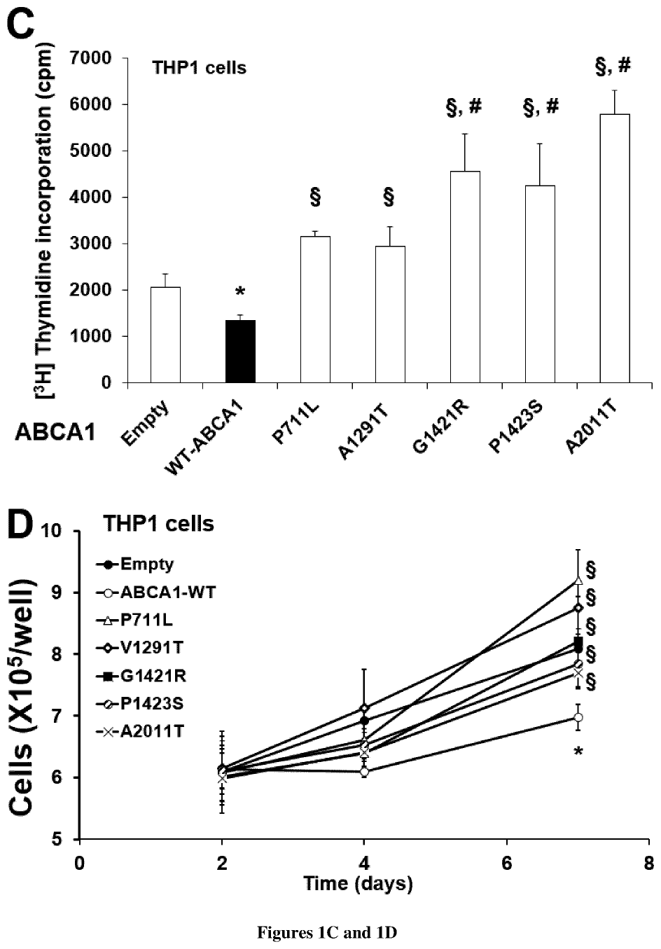

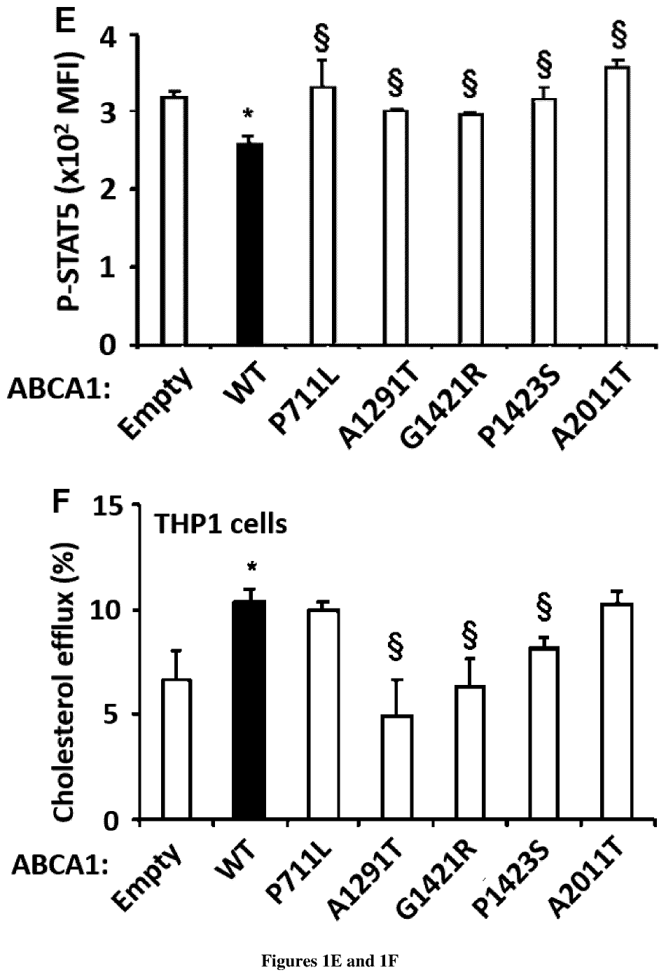

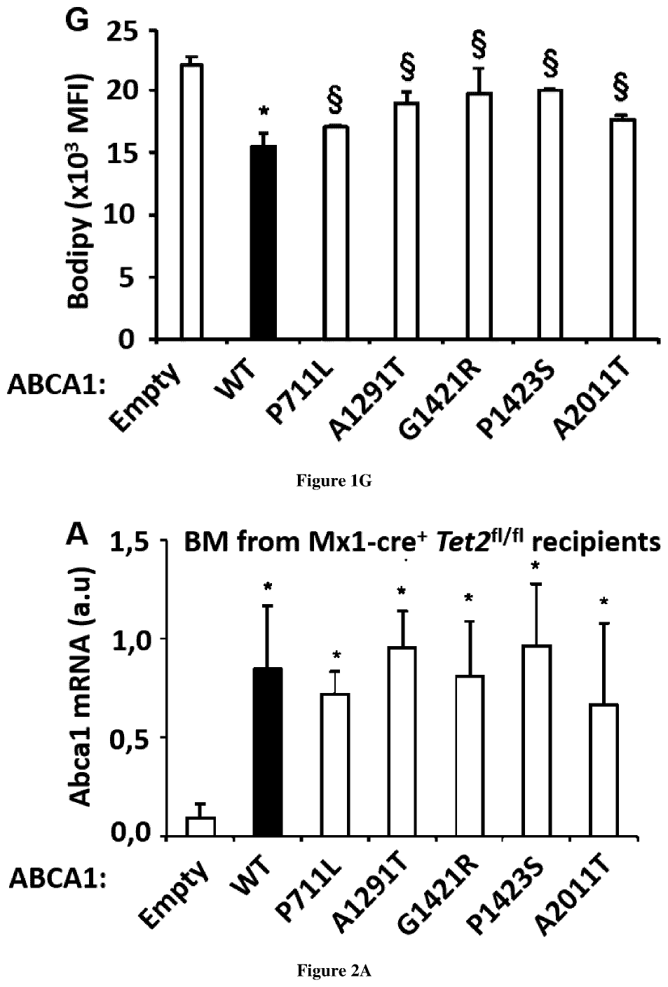

Given the key role of ABCA1-dependent cholesterol efflux pathway in controlling myeloid expansion (Tall and Yvan-Charvet, 2015), we sought to test whether ABCA/CMML mutations affect cellular proliferation. We used site-directed mutagenesis to introduce each of these five somatic mutations individually into the ABCA1 cDNA. To compare the ability of ABCA1 mutants to control proliferation, we transiently transfected HEK293 cells with the ABCA1 cDNAs. Overexpression of WT-ABCA1 resulted in an approximately 1.7-fold decrease in cell proliferation compared with empty vector-transfected cells (data not shown). All mutants located in either the N- and C-terminal regions (P711L and A2011T), the PEST sequence (A1291T) or the apoA-I binding region (G1421R and P1423S) exhibited a significant reduction in anti-proliferative activity (FIG. 1A and data not shown). We also tested the relevance of these ABCA1 mutations in human THP-1 monocytic leukemia cell lines which express endogenous ABCA1 (FIG. 1B). ABCA1-P711L, ABCA1-A129 IT, ABCA1-G1421R, ABCA1-P1423S and ABCA1-A201IT displayed reductions in anti-proliferative activity, compared to ABCA1-WT in lentivirus transduced cells (FIG. 1C), consistent with observations in HEK293 cells. Although the proliferation rate of THP1 cells was slowed down by transient transfection, we showed a growth advantage of all mutations over a culture period of a week compared to WT-ABCA1 expression (FIG. 1D). Stable cell lines expressing ABCA1-G1421R and ABCA1-A201IT (data not shown) with normal proliferation rate showed growth and proliferative advantages compared to ABCA1-WT (data not shown). We also observed an activation of JAK-STAT signaling pathway in ABCA1 mutant-transduced cells compared to WT-ABCA1 as illustrated by higher levels of pSTAT5 quantified by flow cytometry (FIG. 1E). Because assessment of cholesterol efflux capacity is not practical in suspension cells, we next tested the dependence of ABCA1 mutations on their efflux capacity in differentiated THP-1 macrophages. Under this setting, three out of the five ABCA1 mutations (ABCA1-A129 IT, ABCA1-G1421R, ABCA1-P1423S) showed a decrease in the ability of apoA-1 to promote cholesterol efflux compared with WT-ABCA1 (FIG. 1F). Nevertheless, quantification of BODIPY (bore-dipyrromethene)-neutral lipid staining revealed higher neutral lipid accumulation in ABCA1 mutant-transduced cells compared to WT-ABCA1 (FIG. 1G). Quantification of cellular cholesterol content confirmed these findings (data not shown). The increased proliferation of these cells was also associated with increased cholera toxin subunit B (CTx-B) staining of ABCA1 mutant-transduced cells compared to WT-ABCA1 at the cell surface (ABCA1-A129 IT, ABCA1-G1421R, ABCA1-P1423S) or in intracellular endosomal-like structure (ABCA1-P711L, ABCA1-A20117) (data not shown)suggesting increased formation of cholesterol-rich lipid raft or perturbed intracellular cholesterol trafficking (Dietrich et al., 2001).

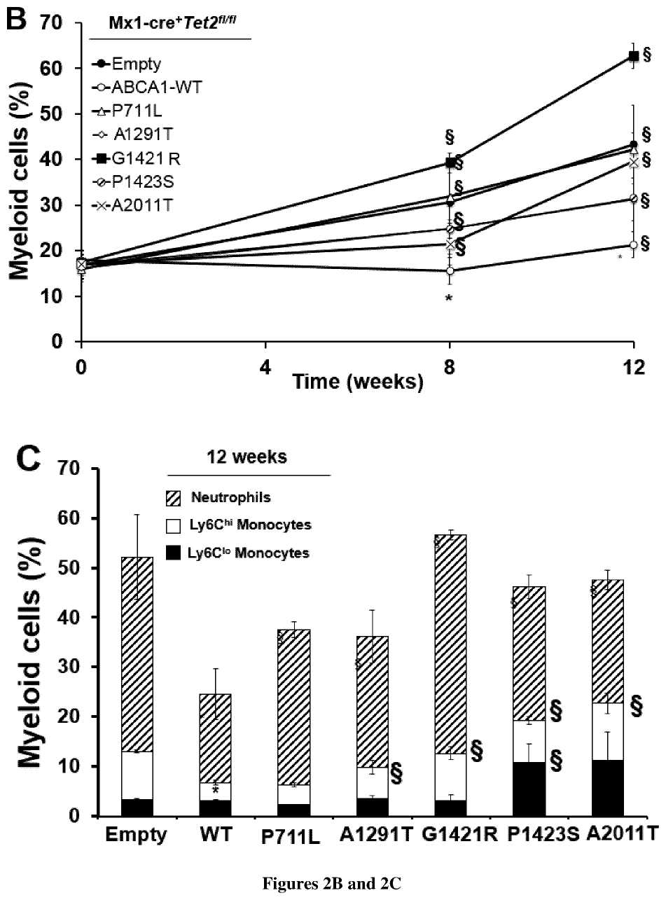

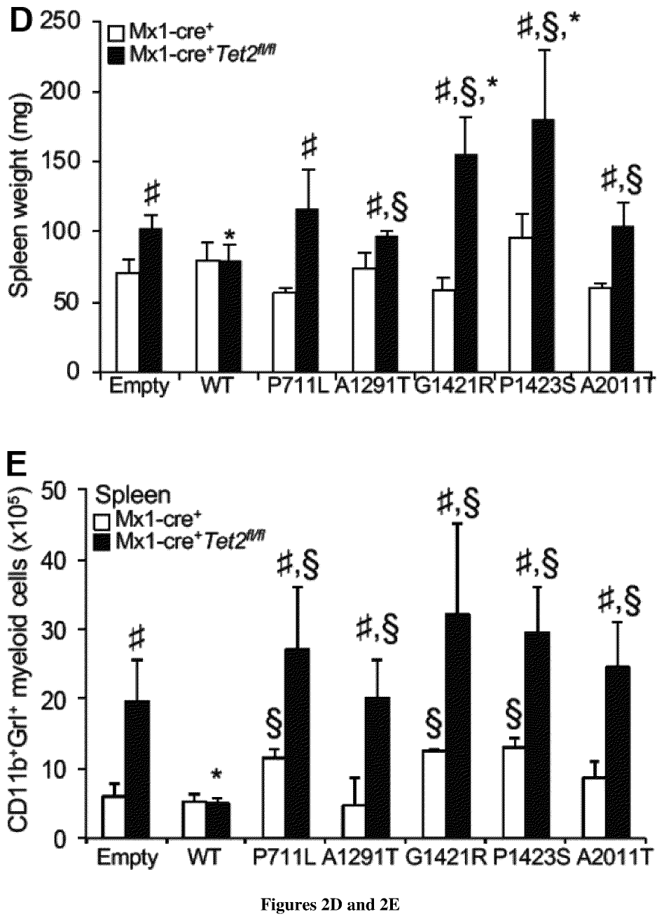

ABCA1 mutants associated with CMML fail to suppress myelopoiesis in vivo. Previous studies have suggested that loss of ABCA1 function alone is insufficient to promote prominent myelopoiesis in hypercholesterolemic mice (Yvan-Charvet et al., 2010). We hypothesized that proliferative effects of ABCA1 mutants observed in CMML might become more evident when combined with other CMML mutant alleles. Tet2 inactivation through loss-of-function mutation is commonly found in CMML (Bowman and Levine, 2017; Solary et al., 2014). Therefore, to assess the in vivo effects of ABCA1 mutants, bone marrow (BM) cells from WT or Mx1-Cre⁺Tet2^{fl/fl} mice (i.e, mice bearing the conditional Tet2 allele and the interferon inducible Cre transgene) were transduced with pLKO-Puro-GFP lentiviral vectors containing WT-ABCA1 or ABCA1 mutants and transplanted into lethally irradiated C57BL/6J mice (data not shown). Animals were analyzed 5 weeks after BM reconstitution (T0) and at the indicated time point following polyinosinic:polycytidylic acid (PIPC) injection (data not shown). Consistent with earlier works (Moran-Crusio et al., 2011; Quivoron et al., 2011), we observed loss of Tet2 expression in the BM of WT recipient mice transplanted with Mx1-Cre⁺Tet2^{fl/fl} BM compared to Mx1-Cre⁺ BM (data not shown) and ablation of the gene was paralleled by a significant reduction of 5-hydroxylation of methycytosine (5hmC) in a pool of peripheral blood cells, which reflect the enzymatic activity of TET2 (data not shown). Quantification of Abca1 mRNA expression confirmed similar levels of overexpression of ABCA1-WT and mutants in the BM of Mx1-Cre⁺Tet2^{fl/fl} recipients (FIG. 2A) and controls (data not shown). Despite similar leukocyte counts (data not shown), overexpression of WT-ABCA1 on a Tet2 deficient background caused a marked reduction in myeloid cells (Gr-1^{high}CD11b^{high}) in blood over time (FIG. 2B) reflecting mainly lower inflammatory Ly6C^{hi} monocyte and neutrophil counts (FIG. 2C and data not shown). In contrast, ABCA1 mutants-expressing animals on a Tet2 deficient background exhibited higher peripheral myeloid cells (both monocytes and neutrophils) compared to ABCA1-WT-transduced animals (FIG. 2B, 2C and data not shown). These effects were not observed when ABCA1-WT or mutants were transduced on a WT background (data not shown). T- and B-cell numbers and hematocrit and platelet counts were normal on both backgrounds (data not shown). These data indicate that ABCA1 mutants impede the protective effect of ABCA1-WT in preventing myeloid expansion on a Tet2 deficient background. Importantly, the five ABCA1 mutations identified in CMML patients were found to be loss-of-function mutations as demonstrated by their failure to suppress blood leukocyte counts in the setting of Tet2 deficiency.

ABCA1 Mutants Fail to Prevent CMML-Associated Extramedullary Hematopoiesis and Splenomegaly.

Overexpression of ABCA1-WT suppressed the splenomegaly of animals transplanted with Tet2 deficient BM (FIG. 2D data not shown). Despite a variability within the groups, this was statistically not observed in ABCA1 mutant-transduced animals on a Tet2 deficient background (FIG. 2D). Pathological examination revealed robust infiltration of myeloid cells, including significant destruction of normal spleen architecture in ABCA1 mutant-transduced animals on a Tet2 deficient background compared to ABCA1-WT transduced animals, similar to what was observed in empty vector-transduced animals (data not shown). Flow analysis of the spleen confirmed an increased proportion and number of CD11b⁺GrI⁺ myeloid cells in ABCA1 mutant-transduced animals on a Tet2 deficient background and to some extent on a WT background compared to ABCA1-WT-transduced animals (FIG. 2E and data not shown). An increased percentage of hematopoietic stem/progenitor cells (LSK cells, Lineage⁻Sca1⁺ c-Kit⁺) was also observed in the spleen of ABCA1 mutant-transduced animals compared to ABCA1-WT-transduced animals (data not shown). Thus, unlike WT ABCA1, specific ABCA1 mutants associated with human CMML were unable to limit the increased extramedullary hematopoiesis and splenomegaly that are classical features of CMML.

ABCA1 Mutants Fail to Suppress Expansion and Myeloid Bias of Tet2 Deficient HSPCs

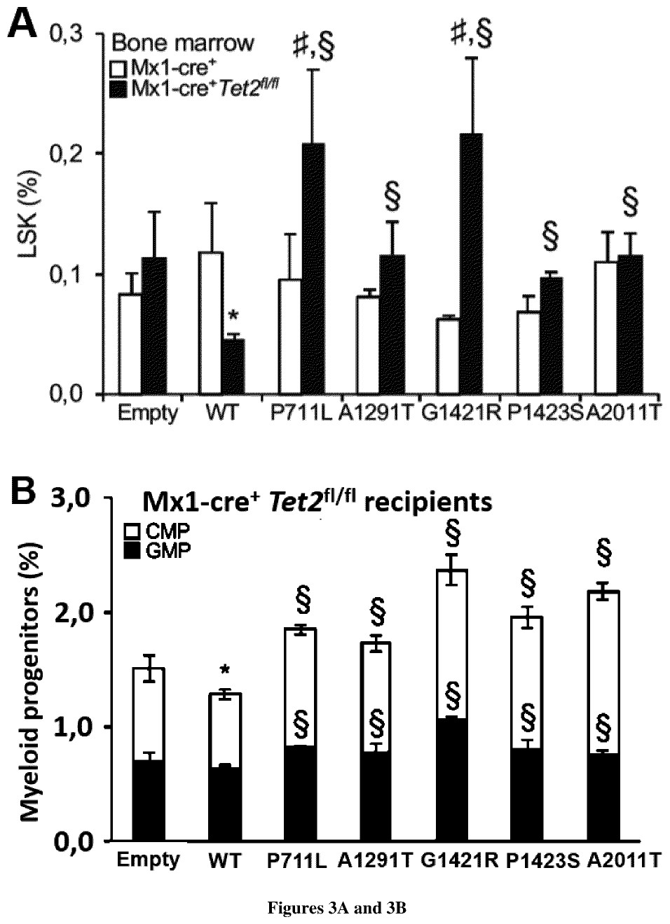

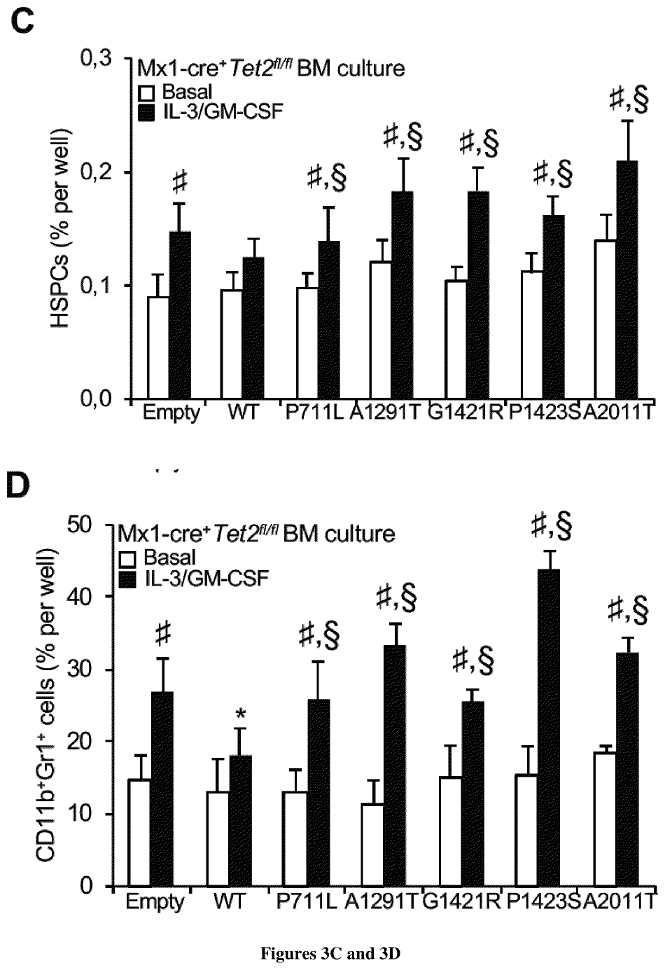

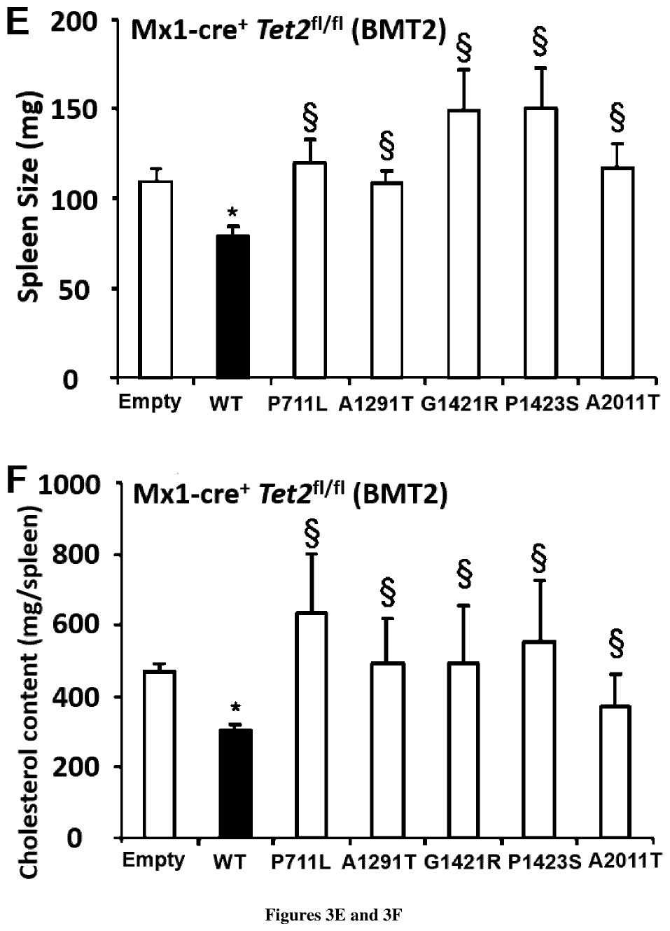

Tet2 loss or defective cholesterol efflux pathways leads to BM hematopoietic stem/progenitor cell expansion (HSPCs) and differentiation toward a myeloid lineage fate in vivo (Moran-Crusio et al., 2011; Quivoron et al., 2011; Yvan-Charvet et al., 2010). Analysis of the BM HSPCs showed a reduction of the LSK cells in ABCA1-WT-transduced animals on a Tet2 deficient background compared to empty control-transduced animals. This effect was lost in ABCA1 mutant-transduced animals (FIG. 3A). Although ABCA1 mutants barely altered the percentage of BM megakaryocyte-erythrocyte progenitors (MEPs, Lineage⁻Sca1⁻ c-Kit⁺CD34^{low}FcγR^{low}) (data not shown), the granulocyte-monocyte progenitors (GMPs, Lineage⁻ Sca1⁻ c-Kit⁺CD34^{hi}FcγR^{hi}) and the common myeloid progenitors (CMPs, Lineage⁻ Sca1⁻ c-Kit⁺CD34^{hi}FcγR^{hi}) were significantly increased in ABCA1 mutant-transduced BM compared to ABCA1-WT-transduced BM both on WT (data not shown) and Tet2 deficient background (FIG. 3B). To determine whether ABCA1 mutants engage HSPCs to differentiate into the myelomonocytic lineages, BM cells from ABCA1 mutant-transduced animals were cultured ex vivo in the presence of IL-3 and GM-CSF. BM cultures showed that ABCA1 mutants disengaged the suppressive effects of ABCA1 on HSPC expansion (FIG. 3C) and myeloid lineage expansion (FIG. 3D) in the Tet2 deficient background. These findings suggest that in contrast to ABCA1-WT, ABCA1 mutants allow Tet2 deficient HSPCs to proliferate and to favor myelopoiesis as a result of increased IL-3/GM-CSF signaling. Of note, in secondary transplants (data not shown), ABCA1 mutant-transduced animals had a significantly higher percentage of peripheral Gr-1^{high}CD11b^{high} myeloid cells compared to ABCA1-WT-transduced animals (data not shown) and failed to suppress splenomegaly (FIG. 3E) or splenic cholesterol accumulation (FIG. 3F). These findings confirm that ABCA1 inactivation not only confers a significant competitive advantage to Tet2 deficient HSPCs in vivo but also a transplantable myeloproliferative disorder.

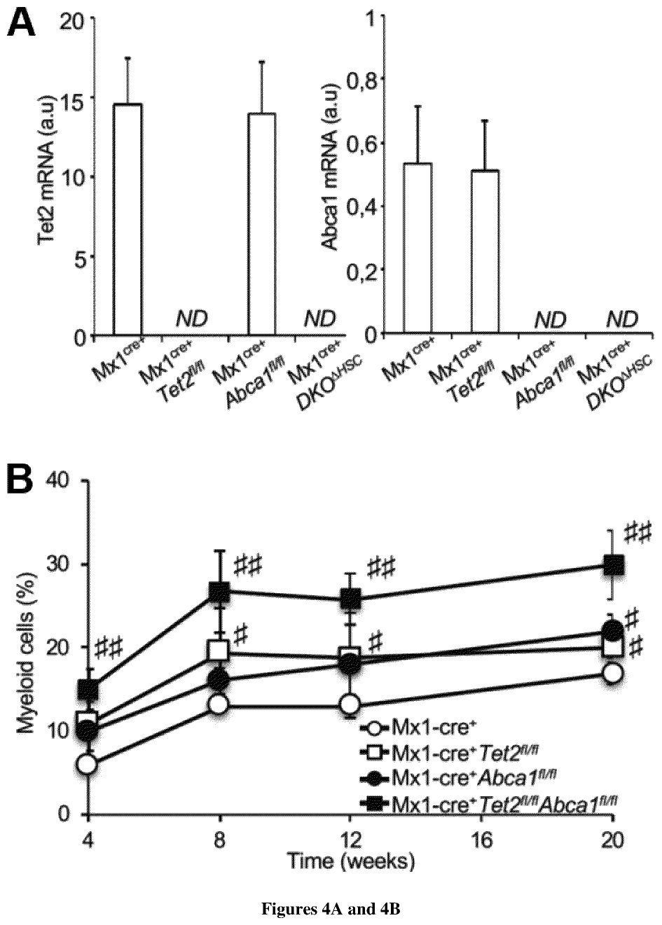

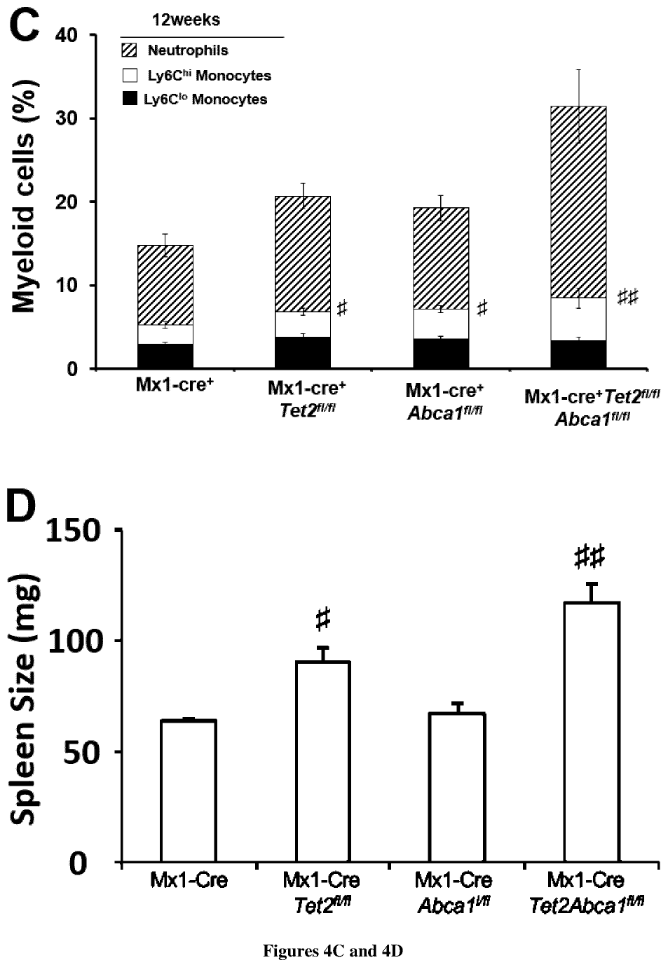

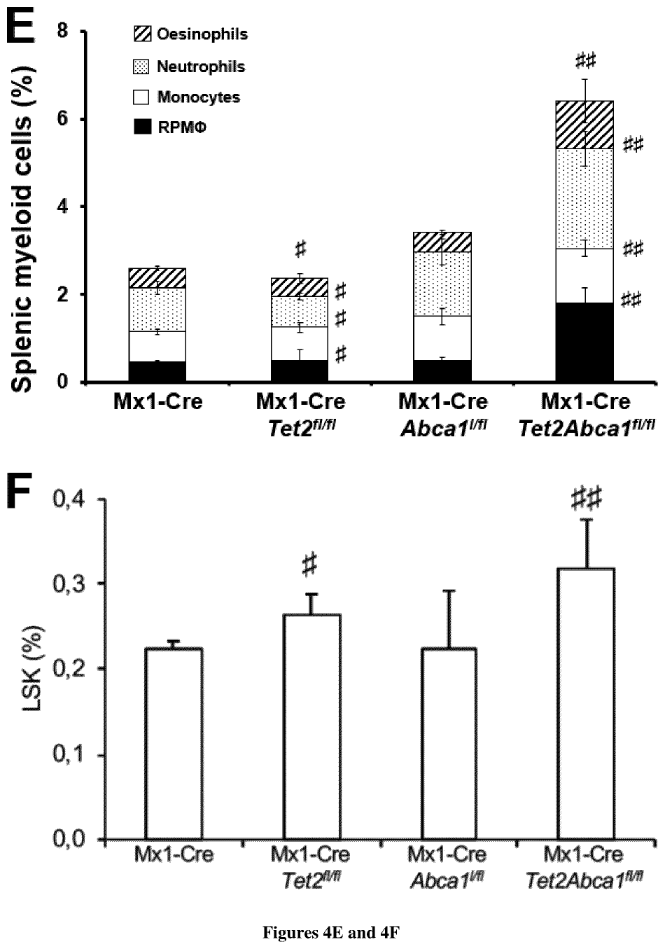

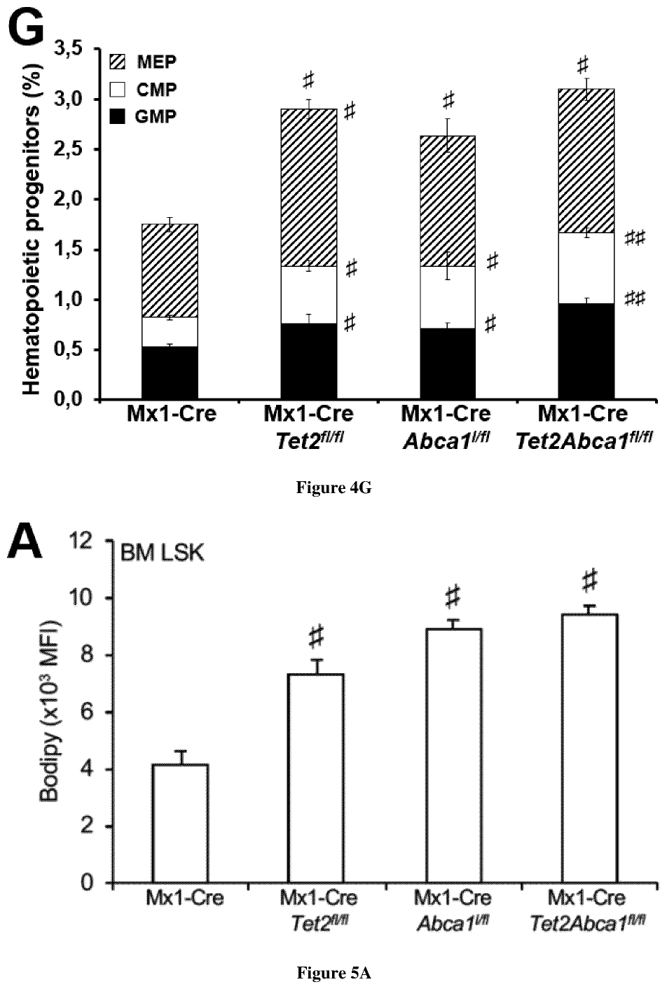

ABCA1 deficiency cooperate with Tet2 loss to propagate myeloid transformation In parallel, we crossed Mx1-Cre⁺Tet2^{fl/fl} mice (Moran-Crusio et al., 2011; Quivoron et al., 2011) to Abca1^{fl/fl} mice (Yvan-Charvet et al., 2010) to generate Mx1-Cre⁺Tet2^{fl/fl}Abca1^{fl/fl} (referred to subsequently as DKO^{ΔHSC}) mice. BM cells from these mice were subsequently transplanted into lethally irradiated C57BL/6J mice (data not shown). Animals were analyzed 5 weeks after BM reconstitution (T0) and at the indicated time point following polyinosinic:polycytidylic acid (PIPC) injection (data not shown). We confirmed the excision of both Tet2 and Abca1 mRNA expression in the BM of Mx1-Cre⁺Tet2^{fl/fl} mice and Mx1-Cre⁺Abca1^{fl/fl} mice, respectively (FIG. 4A). DKO^{ΔHSC} mice also had a marked increase in peripheral myeloid cells compared to single Abca1 or Tet2 deficient mice (FIG. 4B, 4C and data not shown). These findings show a cooperative effect between ABCA1 and Tet2 mutations on myeloid expansion. Additionally, Abca1 deficiency exacerbated the splenomegaly of Tet2-deficient animals while having no effect on its own. (FIG. 4D and data not shown). DKO^{ΔHSC} mice also exhibited higher myeloid cell infiltration compared to single knockout mice after pathological examination (data not shown) with an increased percentage and number of eosinophil, neutrophil, monocyte and red pulp macrophages (RPMs) determined by flow cytometry (FIG. 4E and data not shown). Thus complete deletion of ABCA1 in Tet2-deficient mice increased spleen size and enhanced extramedullary myelopoiesis. Consistently, DKO^{ΔHSC} mice showed an increased frequency of LSK cells compared with WT or Tet2 deficient mice (FIG. 4F). There was also an additive effect of ABCA1 and TET2 deficiency in promoting BM myeloid progenitor expansion (FIG. 4G). Altogether, these data indicate that concurrent ABCA1 deficiency and Tet2 loss in hematopoietic cells exacerbated myeloid transformation supporting the finding that ABCA1 mutations identified in CMML patients were loss-of-function mutations.

Cholesterol Accumulation Links ABCA1 Mutants and Tet2 Loss to IL3-Receptor Signaling Hypersensitivity

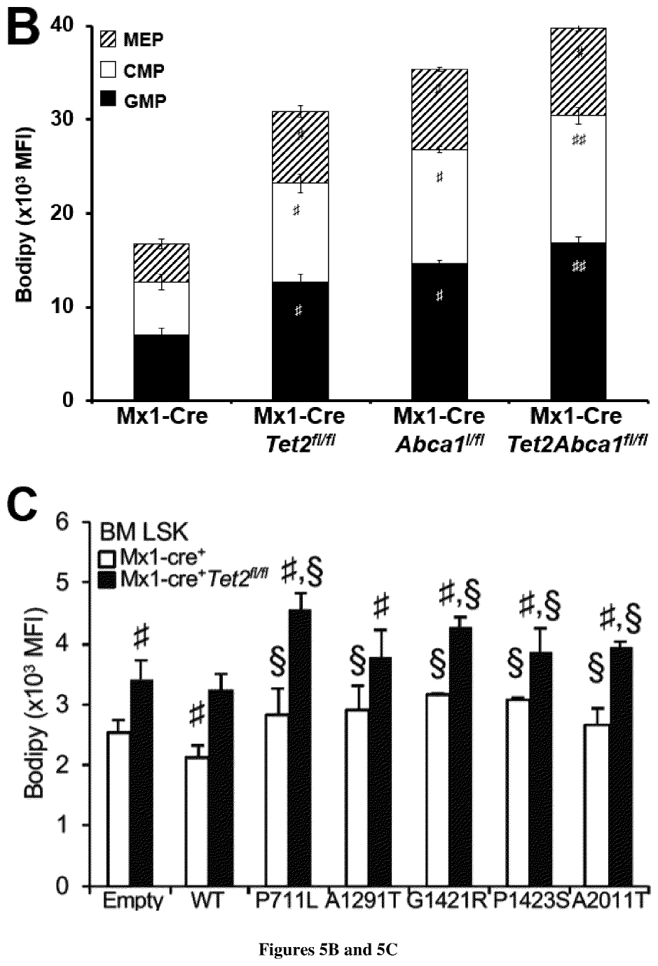

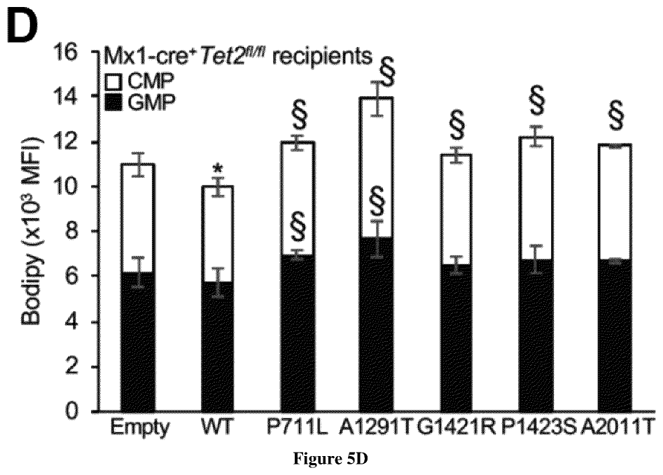

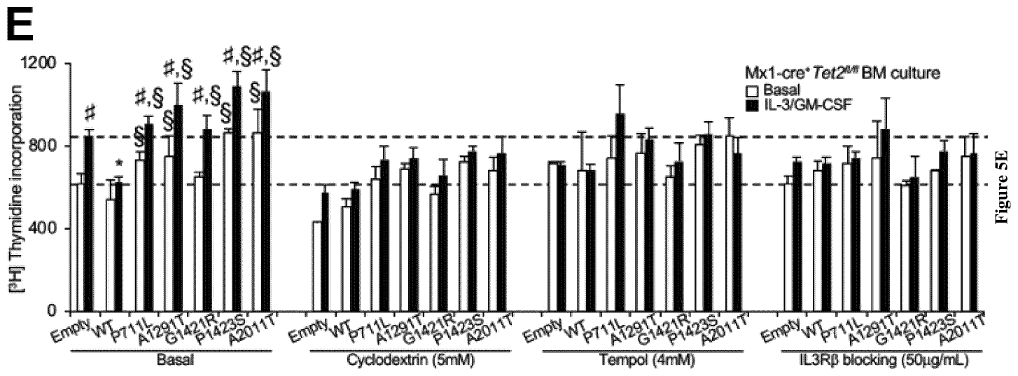

We next sought to identify mechanisms responsible for the lack of tumor suppressor function of ABCA1 mutants in Tet2 deficient BM cells. Increased cholesterol accumulation and reduced expression of ABCA1 have been repeatedly observed in cancer cells (Bovenga et al., 2015; Lin and Gustafsson, 2015). Thus, taking advantage of publicly available gene expression datasets (Kunimoto et al., 2018), we first interrogated whether Tet2 deficient LSK, CMP and GMPs cells could transcriptionally regulate cholesterol metabolic pathways. We didn't observe major transcriptional regulation of genes involved in cholesterol metabolism (<10% overall changes) including liver X receptor (LXR) target genes and ABCA1 in Tet2 deficient hematopoietic progenitors (data not shown). The functional behavior of these cells was next assessed by quantifying neutral lipid accumulation in single knockout and DKO^{ΔHSC} HSPCs by flow cytometry using BODIPY staining. An increase in cellular neutral lipid content in single knockout and DKO^{ΔHSC} HSPCs and committed myeloid progenitors (i.e, GMP and CMP) was observed compared to controls (FIGS. 5A and 5B). Combined deficiency of Tet2 and Abca1 also led to a cumulative BODIPY-neutral lipid staining per cell (expressed as mean fluorescence intensity) in myeloid progenitors compared to single knockout cells (FIG. 5B) but not in HSPCs (FIG. 5A). Consistently, overexpression of WT-ABCA1 reduced BODIPY staining in Tet2 deficient CMP progenitors but not in Tet2 deficient HSPCs (FIGS. 5C and 5D). However, ABCA1 mutants failed to reduce BODIPY staining on a Tet2 deficient background (FIGS. 5C and 5D) and to some extent on a control background (i.e, ABCA1-A1291T, G1421R and P1423S) (data not shown). The anti-tumor activity of ABCA1 could be linked to inhibition of SREBP-2 and cholesterol biosynthesis target genes that has recently been linked to reduced expression of cell growth facilitating mevalonate products in a solid tumor (Moon et al., 2018). However, the accumulation of cholesterol in ABCA1 mutants-transduced Tet2 deficient BM cells was rather associated with reduced expression of genes in the mevalonate pathway (data not shown), which is a well-known feed-back regulatory mechanism by which sterols accumulation in the ER limit SREBP-2 processing and expression of its target genes (Brown and Goldstein, 2009; Spann and Glass, 2013).

Given the activation of the IL3-receptor β canonical pathway in myeloid cells with defective cholesterol efflux pathway (Yvan-Charvet et al., 2010) and the hypersensitivity of Tet2 deficient myeloid cells to GM-CSF (Kunimoto et al., 2018), we next assessed whether the myelo-suppressive function of ABCA1 on a Tet2 deficient background could be attributed to its role in removing excess cellular cholesterol in committed myeloid progenitors. Excess cellular cholesterol was removed by treatment with cyclodextrin in ABCA1 mutant-transduced Tet2 deficient BM cells cultured in presence or absence of IL-3 and GM-CSF. We first validated our ex vivo BM culture proliferation assay by showing that inhibition of the IL-3Rβ signaling pathway using IL-3Rβ blocking antibody prevented BM proliferation on IL-3 and GM-CSF treatment (FIG. 5E). We next showed that both IL-3Rβ blocking antibody and cyclodextrin prevented the proliferation of ABCA1 mutant-transduced Tet2 deficient BM cells similar to the effect of ABCA1-WT overexpression (FIG. 5E), without affecting cell viability (data not shown). Consistently, Western blot analysis showed higher levels of pErk1/2 or pJak2 in ABCA1 mutant-transduced Tet2 deficient BM cells compared to ABCA1-WT-transduced cells (data not shown). mRNA expression analysis also revealed higher cyclin D1 and PU.1 gene expression in ABCA1 mutant-transduced Tet2 deficient BM cells (data not shown). These genes are IL-3Rβ signaling targets involved in proliferation and myeloid lineage commitment. In contrast, we did not observe changes in the expression of the key transcription factor C/EBPα or in negative regulators of RAS signaling including dual specificity phosphatase 1 (DUSP1) or sprouty-related, EEVH1 domain-containing protein 1 (Spred1) (Kunimoto et al., 2018). These findings suggest that the tumor suppressor function of ABCA1 relies on its capacity to modulate cholesterol-dependent IL-3Rβ signaling and downstream MAPK and JAK2 signaling. We and others recently showed that reduced autophagy and enhanced mitochondrial reactive oxygen species (ROS) production were two major metabolic events promoting HSPC expansion and myeloid commitment downstream of the IL-3Rβ signaling pathway (Lum et al., 2005; Sarrazy et al., 2016). Thus, we estimated autophagic activity using the Cyto-ID probe by flow cytometry. Reduced autophagic activity was observed in Tet2 and Abca1 deficient myeloid progenitors (data not shown) and also in ABCA1 mutant-transduced CMP and GMP cells compared to ABCA1-WT-transduced BM both on WT and Tet2 deficient (data not shown). Finally, suppression of mitochondrial ROS with tempol prevented the basal proliferation and IL-3/GM-CSF hypersensitivity of ABCA1 mutants-transduced Tet2 deficient BM cells (FIG. 5E). These data confirm that ABCA1 mutations impair the myelo-suppressive function of ABCA1 by increasing the IL-3/GM-CSF hypersensitivity and downstream metabolic sequelae in Tet2 deficient myeloid progenitors.

Increased HDL Reverses Increased Myelopoiesis and Splenomegaly Caused by ABCA1 Mutants

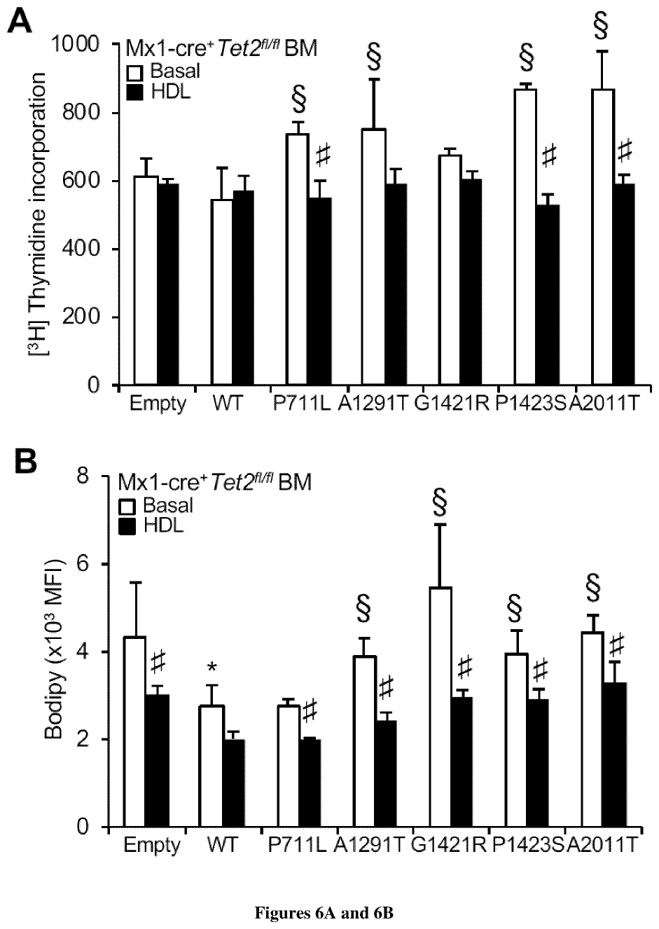

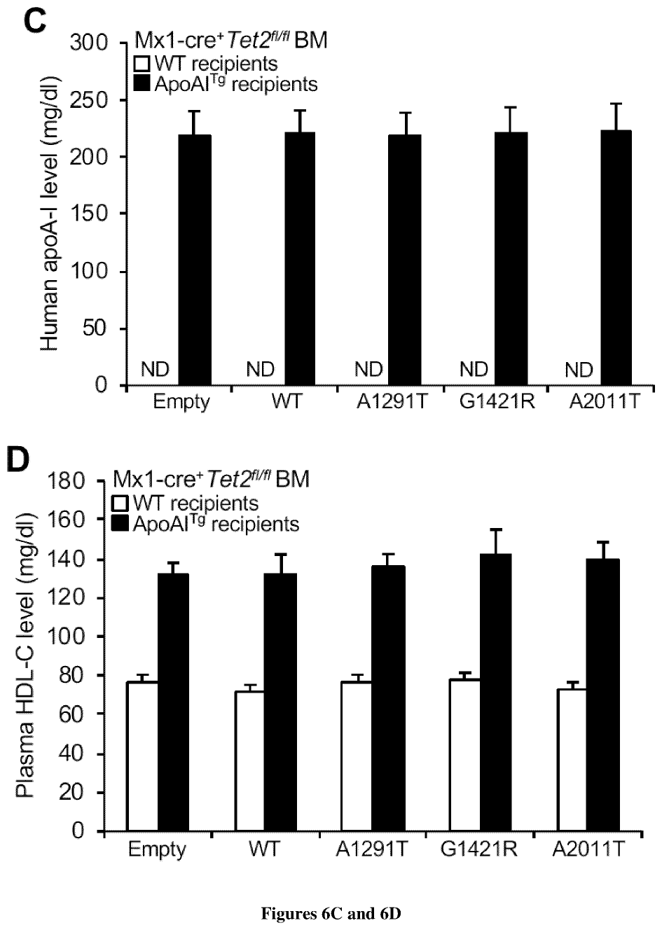

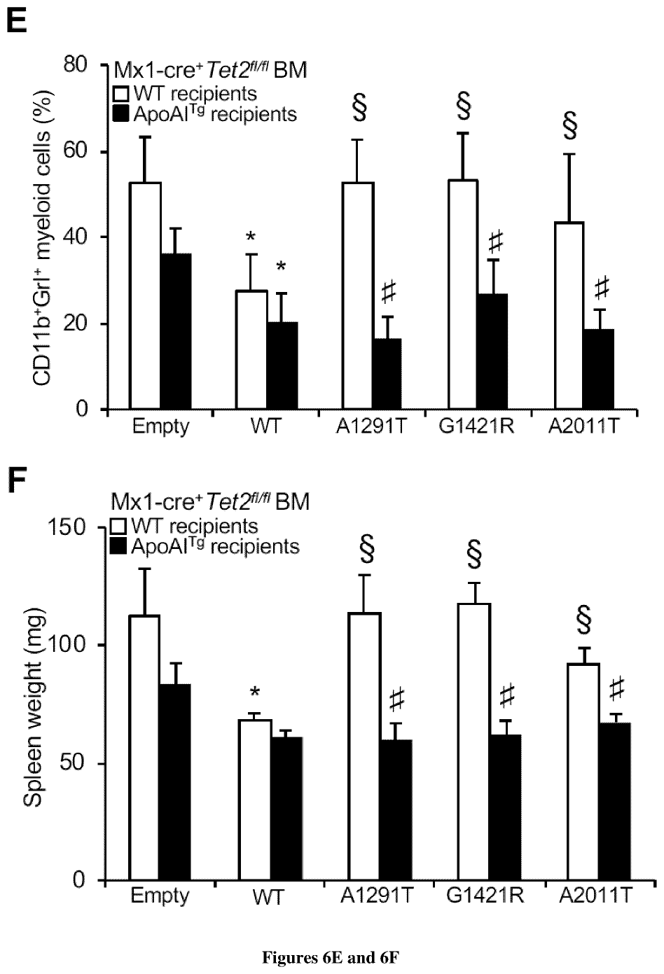

Given the ability of increased HDL to suppress HSPC myeloid lineage commitment and rescue the myeloproliferative disorder of mice with defective cholesterol efflux (Yvan-Charvet et al., 2010), we hypothesized that raising HDL would alleviate some of the myeloproliferative phenotypes of ABCA1 mutant-transduced Tet2 deficient animals. First, HDL treatment ex vivo reduced the proliferation rates (FIG. 6A) and BODIPY cholesterol staining (FIG. 6B) of ABCA1 mutant-transduced Tet2 deficient BM cells. We next explored the efficacy of the apoA-1 transgene to increase HDL levels in vivo (FIGS. 6C and 6D). Transduction of ABCA1-A1291T, G1421R and A2011T were selected as proof of concept and compared to BM transduced with empty or ABCA1-WT. Similar to ABCA1-WT-transduced BM cells, the human apoA-1 transgene prevented the peripheral myeloid expansion of mice transplanted with ABCA1 mutants-transduced Tet2 deficient BM (FIG. 6E) and also abolished their splenomegaly (FIG. 6F). These data demonstrate that increased apoA-1 production and increased HDL show robust preclinical efficacy in leukemia driven by ABCA1 and TET2 mutants.

### REFERENCES

Throughout this application, various references describe the state of the art to which this invention pertains. The disclosures of these references are hereby incorporated by reference into the present disclosure.
1. Cancer Genome Atlas Research Network, T. J. Ley, C. Miller, L. Ding, B. J. Raphael, A. J. Mungall, A. G. Robertson, K. Hoadley, T. J. Triche, P. W. Laird, J. D. Baty, L. L. Fulton, R. Fulton, S. E. Heath, J. Kalicki-Veizer, C. Kandoth, J. M. Klco, D. C. Koboldt, K.-L. Kanchi, S. Kulkarni, T. L. Lamprecht, D. E. Larson, L. Lin, C. Lu, M. D. McLellan, J. F. McMichael, J. Payton, H. Schmidt, D. H. Spencer, M. H. Tomasson, J. W. Wallis, L. D. Wartman, M. A. Watson, J. Welch, M. C. Wendl, A. Ally, M. Balasundaram, I. Birol, Y. Butterfield, R. Chiu, A. Chu, E. Chuah, H.-J. Chun, R. Corbett, N. Dhalla, R. Guin, A. He, C. Hirst, M. Hirst, R. A. Holt, S. Jones, A. Karsan, D. Lee, H. I. Li, M. A. Marra, M. Mayo, R. A. Moore, K. Mungall, J. Parker, E. Pleasance, P. Plettner, J. Schein, D. Stoll, L. Swanson, A. Tam, N. Thiessen, R. Varhol, N. Wye, Y. Zhao, S. Gabriel, G. Getz, C. Sougnez, L. Zou, M. D. M. Leiserson, F. Vandin, H.-T. Wu, F. Applebaum, S. B. Baylin, R. Akbani, B. M. Broom, K. Chen, T. C. Motter, K. Nguyen, J. N. Weinstein, N. Zhang, M. L. Ferguson, C. Adams, A. Black, J. Bowen, J. Gastier-Foster, T. Grossman, T. Lichtenberg, L. Wise, T. Davidsen, J. A. Demchok, K. R. M. Shaw, M. Sheth, H. J. Sofia, L. Yang, J. R. Downing, G. Eley, Genomic and epigenomic landscapes of adult de novo acute myeloid leukemia, N. Engl. J. Med. 368, 2059-2074 (2013).
2. T. Sjöblom, S. Jones, L. D. Wood, D. W. Parsons, J. Lin, T. D. Barber, D. Mandelker, R. J.

Leary, J. Ptak, N. Silliman, S. Szabo, P. Buckhaults, C. Farrell, P. Meeh, S. D. Markowitz, J. Willis, D. Dawson, J. K. V. Willson, A. F. Gazdar, J. Hartigan, L. Wu, C. Liu, G. Parmigiani, B. H. Park, K. E. Bachman, N. Papadopoulos, B. Vogelstein, K. W. Kinzler, V. E. Velculescu, The consensus coding sequences of human breast and colorectal cancers, Science 314, 268-274 (2006).
3. A. H. Shih, Y. Jiang, C. Meydan, K. Shank, S. Pandey, L. Barreyro, I. Antony-Debre, A. Viale, N. Socci, Y. Sun, A. Robertson, M. Cavatore, E. de Stanchina, T. Hricik, F. Rapaport, B. Woods, C. Wei, M. Hatlen, M. Baljevic, S. D. Nimer, M. Tallman, E. Paietta, L. Cimmino, I. Aifantis, U. Steidl, C. Mason, A. Melnick, R. L. Levine, Mutational cooperativity linked to combinatorial epigenetic gain of function in acute myeloid leukemia, Cancer Cell 27, 502-515 (2015).
4. H. Kunimoto, C. Meydan, A. Nazir, J. Whitfield, K. Shank, F. Rapaport, R. Maher, E. Pronier, S. C. Meyer, F. E. Garrett-Bakelman, M. Tallman, A. Melnick, R. L. Levine, A. H. Shih, Cooperative Epigenetic Remodeling by TET2 Loss and NRAS Mutation Drives Myeloid Transformation and MEK Inhibitor Sensitivity, Cancer Cell 33, 44-59.e8 (2018).
5. L. Busque, J. P. Patel, M. Figueroa, A. Vasanthakumar, S. Provost, Z. Hamilou, L. Mollica, J. Li, A. Viale, A. Heguy, M. Hassimi, N. Socci, P. K. Bhatt, M. Gonen, C. E. Mason, A. Melnick, L. A. Godley, C. Brennan, 0. Abdel-Wahab, R. L. Levine, Recurrent Somatic TET2 Mutations in Normal Elderly Individuals With Clonal Hematopoiesis, Nat Genet 44, 1179-1181 (2012).
6. H. Jafri, A. A. Alsheikh-Ali, R. H. Karas, Baseline and on-treatment high-density lipoprotein cholesterol and the risk of cancer in randomized controlled trials of lipid-altering therapy, J. Am. Coll. Cardiol. 55, 2846-2854 (2010).
7. A. R. Tall, L. Yvan-Charvet, Cholesterol, inflammation and innate immunity, Nat. Rev. Immunol. 15, 104-116 (2015).
8. F. Bovenga, C. Sabbà, A. Moschetta, Uncoupling nuclear receptor LXR and cholesterol metabolism in cancer, Cell Metab. 21, 517-526 (2015).
9. J. W. Clendening, L. Z. Penn, Targeting tumor cell metabolism with statins, Oncogene 31, 4967-4978 (2012).
10. C. V. Dang, Links between metabolism and cancer, Genes Dev. 26, 877-890 (2012).
11. C.-Y. Lin, J.-Å. Gustafsson, Targeting liver X receptors in cancer therapeutics, Nat. Rev. Cancer 15, 216-224 (2015).
12. P. J. Mullen, R. Yu, J. Longo, M. C. Archer, L. Z. Penn, The interplay between cell signalling and the mevalonate pathway in cancer, Nat. Rev. Cancer 16, 718-731 (2016).
13. A. J. Murphy, M. Akhtari, S. Tolani, T. Pagler, N. Bijl, C.-L. Kuo, M. Wang, M. Sanson, S. Abramowicz, C. Welch, A. E. Bochem, J. A. Kuivenhoven, L. Yvan-Charvet, A. R. Tall, ApoE regulates hematopoietic stem cell proliferation, monocytosis, and monocyte accumulation in atherosclerotic lesions in mice, J. Clin. Invest. 121, 4138-4149 (2011).
14. M. Wang, M. Subramanian, S. Abramowicz, A. J. Murphy, A. Gonen, J. Witztum, C. Welch, I. Tabas, M. Westerterp, A. R. Tall, Interleukin-3/granulocyte macrophage colony-stimulating factor receptor promotes stem cell expansion, monocytosis, and atheroma macrophage burden in mice with hematopoietic ApoE deficiency, Arterioscler. Thromb. Vasc. Biol. 34, 976-984 (2014).
15. M. Westerterp, S. Gourion-Arsiquaud, A. J. Murphy, A. Shih, S. Cremers, R. L. Levine, A. R. Tall, L. Yvan-Charvet, Regulation of hematopoietic stem and progenitor cell mobilization by cholesterol efflux pathways, Cell Stem Cell 11, 195-206 (2012).
16. L. Yvan-Charvet, T. Pagler, E. L. Gautier, S. Avagyan, R. L. Siry, S. Han, C. L. Welch, N. Wang, G. J. Randolph, H. W. Snoeck, A. R. Tall, ATP-binding cassette transporters and HDL suppress hematopoietic stem cell proliferation, Science 328, 1689-1693 (2010).
17. E. L. Gautier, M. Westerterp, N. Bhagwat, S. Cremers, A. Shih, O. Abdel-Wahab, D. Lütjohann, G. J. Randolph, R. L. Levine, A. R. Tall, L. Yvan-Charvet, HDL and Glut1 inhibition reverse a hypermetabolic state in mouse models of myeloproliferative disorders, J. Exp. Med. 210, 339-353 (2013).
18. R. Itzykson, M. Duchmann, N. Lucas, E. Solary, CMML: Clinical and molecular aspects, Int. J. Hematol. 105, 711-719 (2017).
19. L. R. Brunham, R. R. Singaraja, M. R. Hayden, Variations on a gene: rare and common variants in ABCA1 and their impact on HDL cholesterol levels and atherosclerosis, Annu. Rev. Nutr. 26, 105-129 (2006).
20. P. D. Emanuel, Juvenile myelomonocytic leukemia and chronic myelomonocytic leukemia, Leukemia 22, 1335-1342 (2008).
21. R. L. Bowman, R. L. Levine, TET2 in Normal and Malignant Hematopoiesis, Cold Spring Harb Perspect Med 7 (2017), doi:10.1101/cshperspect.a026518.
22. E. Solary, O. A. Bernard, A. Tefferi, F. Fuks, W. Vainchenker, The Ten-Eleven Translocation-2 (TET2) gene in hematopoiesis and hematopoietic diseases, Leukemia 28, 485-496 (2014).
23. K. Moran-Crusio, L. Reavie, A. Shih, O. Abdel-Wahab, D. Ndiaye-Lobry, C. Lobry, M. E. Figueroa, A. Vasanthakumar, J. Patel, X. Zhao, F. Perna, S. Pandey, J. Madzo, C. Song, Q. Dai, C. He, S. Ibrahim, M. Beran, J. Zavadil, S. D. Nimer, A. Melnick, L. A. Godley, I. Aifantis, R. L. Levine, Tet2 loss leads to increased hematopoietic stem cell self-renewal and myeloid transformation, Cancer Cell 20, 11-24 (2011).
24. C. Quivoron, L. Couronné, V. Della Valle, C. K. Lopez, I. Plo, O. Wagner-Ballon, M. Do Cruzeiro, F. Delhommeau, B. Arnulf, M.-H. Stern, L. Godley, P. Opolon, H. Tilly, E. Solary, Y. Duffourd, P. Dessen, H. Merle-Beral, F. Nguyen-Khac, M. Fontenay, W. Vainchenker, C. Bastard, T. Mercher, O. A. Bernard, TET2 inactivation results in pleiotropic hematopoietic abnormalities in mouse and is a recurrent event during human lymphomagenesis, Cancer Cell 20, 25-38 (2011).
25. S.-H. Moon, C.-H. Huang, S. L. Houlihan, K. Regunath, W. A. Freed-Pastor, J. P. Morris, D. F. Tschaharganeh, E. R. Kastenhuber, A. M. Barsotti, R. Culp-Hill, W. Xue, Y.-J. Ho, T. Baslan, X. Li, A. Mayle, E. de Stanchina, L. Zender, D. R. Tong, A. D'Alessandro, S. W. Lowe, C. Prives, p53 Represses the Mevalonate Pathway to Mediate Tumor Suppression, Cell (2018), doi:10.1016/j.ce11.2018.11.011.
26. M. S. Brown, J. L. Goldstein, Cholesterol feedback: from Schoenheimer's bottle to Scap's MELADL, J. Lipid Res. 50 Suppl, S15-27 (2009).
27. N. J. Spann, C. K. Glass, Sterols and oxysterols in immune cell function, Nat. Immunol. 14, 893-900 (2013).
28. J. J. Lum, D. E. Bauer, M. Kong, M. H. Harris, C. Li, T. Lindsten, C. B. Thompson, Growth Factor Regulation of Autophagy and Cell Survival in the Absence of Apoptosis, Cell 120, 237-248 (2005).
29. V. Sarrazy, M. Viaud, M. Westerterp, S. Ivanov, S. Giorgetti-Peraldi, R. Guinamard, E. L. Gautier, E. B. Thorp, D. C. De Vivo, L. Yvan-Charvet, Disruption of Glut1 in Hematopoietic Stem Cells Prevents Myelopoiesis and Enhanced Glucose Flux in Atheromatous Plaques of ApoE(−/−) Mice, Circ. Res. 118, 1062-1077 (2016).
30. L. K. Boroughs, R. J. DeBerardinis, Metabolic pathways promoting cancer cell survival and growth, Nat. Cell Biol. 17, 351-359 (2015).
31. D. Hanahan, R. A. Weinberg, Hallmarks of cancer: the next generation, Cell 144, 646-674 (2011).
32. G. Kroemer, J. Pouyssegur, Tumor cell metabolism: cancer's Achilles' heel, Cancer Cell 13, 472-482 (2008).
33. H. A. Hirsch, D. Iliopoulos, A. Joshi, Y. Zhang, S. A. Jaeger, M. Bulyk, P. N. Tsichlis, X. Shirley Liu, K. Struhl, A transcriptional signature and common gene networks link cancer with lipid metabolism and diverse human diseases, Cancer Cell 17, 348-361 (2010).
34. H. R. McMurray, E. R. Sampson, G. Compitello, C. Kinsey, L. Newman, B. Smith, S.-R. Chen, L. Klebanov, P. Salzman, A. Yakovlev, H. Land, Synergistic response to oncogenic mutations defines gene class critical to cancer phenotype, Nature 453, 1112-1116 (2008).
35. F. Su, K. R. Kozak, S. Imaizumi, F. Gao, M. W. Amneus, V. Grijalva, C. Ng, A. Wagner, G. Hough, G. Farias-Eisner, G. M. Anantharamaiah, B. J. Van Lenten, M. Navab, A. M. Fogelman, S. T. Reddy, R. Farias-Eisner, Apolipoprotein A-I (apoA-I) and apoA-I mimetic peptides inhibit tumor development in a mouse model of ovarian cancer, Proc. Natl. Acad. Sci. U.S.A. 107, 19997-20002 (2010).
36. S. Yang, M. G. Damiano, H. Zhang, S. Tripathy, A. J. Luthi, J. S. Rink, A. V. Ugolkov, A. T. K. Singh, S. S. Dave, L. I. Gordon, C. S. Thaxton, Biomimetic, synthetic HDL nanostructures for lymphoma, Proc. Natl. Acad. Sci. U.S.A. 110, 2511-2516 (2013).
37. M. Zamanian-Daryoush, D. Lindner, T. C. Tallant, Z. Wang, J. Buffa, E. Klipfell, Y. Parker, D. Hatala, P. Parsons-Wingerter, P. Rayman, M. S. S. Yusufishaq, E. A. Fisher, J. D. Smith, J. Finke, J. A. DiDonato, S. L. Hazen, The cardioprotective protein apolipoprotein A1 promotes potent anti-tumorigenic effects, J. Biol. Chem. 288, 21237-21252 (2013).
38. B. Smith, H. Land, Anticancer activity of the cholesterol exporter ABCA1 gene, Cell Rep 2, 580-590 (2012).
39. S. Dessi, B. Batetta, O. Spano, F. Sanna, M. Tonello, M. Giacchino, L. Tessitore, P. Costelli, F. M. Baccino, E. Madon, Clinical remission is associated with restoration of normal high-density lipoprotein cholesterol levels in children with malignancies, Clin. Sci. 89, 505-510 (1995).
40. H. S. Gilbert, H. Ginsberg, Hypocholesterolemia as a manifestation of disease activity in chronic myelocytic leukemia, Cancer 51, 1428-1433 (1983).
41. M. Lacy, D. Atzler, R. Liu, M. de Winther, C. Weber, E. Lutgens, Interactions between dyslipidemia and the immune system and their relevance as putative therapeutic targets in atherosclerosis, Pharmacology & Therapeutics 193, 50-62 (2019).
42. O. Soehnlein, F. K. Swirski, Hypercholesterolemia links hematopoiesis with atherosclerosis, Trends Endocrinol. Metab. 24, 129-136 (2013).
43. M. Tahiliani, K. P. Koh, Y. Shen, W. A. Pastor, H. Bandukwala, Y. Brudno, S. Agarwal, L. M. Iyer, D. R. Liu, L. Aravind, A. Rao, Conversion of 5-methylcytosine to 5-hydroxymethylcytosine in mammalian DNA by MLL partner TET1, Science 324, 930-935 (2009).
44. M. H. Kang, R. Singaraja, M. R. Hayden, Adenosine-triphosphate-binding cassette transporter-1 trafficking and function, Trends Cardiovasc. Med. 20, 41-49 (2010).
45. A. M. Vaughan, C. Tang, J. F. Oram, ABCA1 mutants reveal an interdependency between lipid export function, apoA-I binding activity, and Janus kinase 2 activation, J. Lipid Res. 50, 285-292 (2009).
46. C. Tang, Y. Liu, P. S. Kessler, A. M. Vaughan, J. F. Oram, The macrophage cholesterol exporter ABCA1 functions as an anti-inflammatory receptor, J. Biol. Chem. 284, 32336-32343 (2009).
47. N. Wang, M. Ranalletta, F. Matsuura, F. Peng, A. R. Tall, LXR-Induced Redistribution of ABCG1 to Plasma Membrane in Macrophages Enhances Cholesterol Mass Efflux to HDL, Arterioscler Thromb Vasc Biol 26, 1310-1316 (2006).
48. B. H. Lee, M. G. Taylor, P. Robinet, J. D. Smith, J. Schweitzer, E. Sehayek, S. M. Falzarano, C. Magi-Galluzzi, E. A. Klein, A. H. Ting, Dysregulation of cholesterol homeostasis in human prostate cancer through loss of ABCA1, Cancer Res. 73, 1211-1218 (2013).
49. M. G. Vander Heiden, L. C. Cantley, C. B. Thompson, Understanding the Warburg effect: the metabolic requirements of cell proliferation, Science 324, 1029-1033 (2009).
50. O Abdel-Wahab, R. L. Levine, Metabolism and the leukemic stem cell, J. Exp. Med. 207, 677-680 (2010).
51. M. Pirro, B. Ricciuti, D. J. Rader, A. L. Catapano, A. Sahebkar, M. Banach, High density lipoprotein cholesterol and cancer: Marker or causative?, Progress in Lipid Research 71,54-69 (2018).

1. A method for diagnosing a chronic myelomonocytic leukemia (CMML) in a subject, wherein said method comprising a step of detecting a at least one mutation in a ATP-binding cassette A1 (ABCA1) gene, RNA or protein in a biological sample obtained from said subject, wherein the presence of a mutation is indicative of a CMML.

2. A method for predicting the survival time of a subject suffering from chronic myelomonocytic leukemia (CMML) comprising the steps of i) identifying at least one mutation in ATP-binding cassette A1 (ABCA1) at gene, RNA or protein in a biological sample obtained from the subject; and ii) concluding that the subject will have a short survival time when at least one mutation in ABCA1 is identified or concluding that the subject will have a long survival time when any mutation is not identified in ABCA1.

3. The method according to claim 1, wherein the at least one mutation is ABCA1-P711L, ABCA1-A1291T, ABCA1-G1421R, ABCA1-P1423S and/or ABCA1-A2011T.

4. The method according to claim 1, wherein multiple mutations are identified simultaneously, separately or sequentially.

5. The method according to claim 1, wherein the biological sample is a tissue biopsy.

6. The method according to claim 1, wherein the biological sample is a bone marrow sample.

7. The method according to claim 1, wherein the biological sample is a blood sample.

8. A method for treating a subject suffering from chronic myelomonocytic leukemia (CMML) comprising a step of administering to the subject a therapeutically effective amount of HDL/ABCA recombinant (ApoA-1); cylodextrin and/or anti-IL-3Rbeta antibody.

9. The method according to claim 8 wherein said subject is i) diagnosed as having CMML by detecting, in a biological sample obtained from said subject, at least one mutation in an ATP-binding cassette A1 (ABCA1) gene, RNA or protein, wherein the presence of a mutation is indicative of CMML, and/or ii) identified as having a short survival time by identifying at least one mutation in an ATP-binding cassette A1 (ABCA1) at gene, RNA or protein in the biological sample.

10. A kit for performing the methods of the present invention, wherein said kit comprises means for measuring at least one mutation in ABCA1 protein and/or detecting ABCA1 SNP that is indicative of the risk of having a short survival time in a subject.

11. The method according to claim 8, wherein the at least one mutation is ABCA1-P711L, ABCA1-A1291T, ABCA1-G1421R, ABCA1-P1423S and/or ABCA1-A2011T.

12. The method according to claim 8, wherein multiple mutations are identified simultaneously, separately or sequentially.

13. The method according to claim 8, wherein the biological sample is a tissue biopsy.

14. The method according to claim 8, wherein the biological sample is a bone marrow sample.

15. The method according to claim 8, wherein the biological sample is a blood sample.
